(19) United States
(12) Patent Application Publication
(10) Pub. No.: US 2023/0159698 A1
(43) Pub. Date: May 25, 2023

(54) XYLITOL-DOPED CITRATE COMPOSITIONS AND USES THEREOF

(71) Applicant: THE PENN STATE RESEARCH FOUNDATION, University Park, PA (US)

(72) Inventors: Jian YANG, State College, PA (US); Ethan GERHARD, State College, PA (US)

(21) Appl. No.: 17/917,705

(22) PCT Filed: Apr. 7, 2021
(86) PCT No.: PCT/US2021/026177

§ 371 Date: Oct. 7, 2022

Related U.S. Application Data
(60) Provisional application No. 63/006,521, filed on Apr. 7, 2020.

(51) Int. Cl. C08G 63/78; A61L 27/58; A61L 27/18; A61L 27/36; C08L 67/04; A61L 27/02

(52) U.S. Cl. CPC C08G 63/78; A61L 27/58; A61L 27/18; A61L 27/365; C08L 67/04; A61L 27/02; A61L 2430/02

(57) ABSTRACT

The present disclosure provides compositions which may be used as tissue engineering materials, and more particularly xylitol-doped citrate polymer compositions which may be useful as bone grafts.

### CROSS-REFERENCE TO RELATED APPLICATIONS

This application claims the benefit of priority to U.S. Provisional Application No. 63/006,521, filed Apr. 7, 2020, the disclosure of which is incorporated herein by reference in its entirety.

### TECHNICAL FIELD

This disclosure relates to compositions which may be used as tissue engineering materials, and more particularly to xylitol-doped citrate polymer compositions which may be useful as bone grafts.

### BACKGROUND

The generation of viable and functional bone grafts that replicate the mechanical and osteogenic bioactivity of native bone has marked potential to improve the field of reconstructive orthopedic surgery. Critical for repair and reconstruction of congenital defects, cancer resections and trauma-related injury is the ability to design such grafts with maximal efficiency that replicate the viscoelastic and antifatigue properties as well as the bioactivity of physiological bone tissue. Additionally, the ability to readily tailor the physical and bioactive properties of such materials is highly sought after. In particular, replication of the mechanical properties of native bone while concurrently attaining a degradation rate suitable for tissue in growth remains a major challenge when creating bone grafts. Currently, generation of suitable grafts is limited by the availability of bio-derived materials and the poor mechanical and degradation properties as well as the limited biocompatibility and osteogenic activity of synthetic polymeric materials.

Bone reconstructions often involve the use of allograft or autograft to replace damaged tissue. A significant limitation of these techniques is the difficulty in harvesting materials and of three-dimensional contouring to match the original tissue geometry to be replaced. Additionally, donor site tissue morbidity or incompatibility and disease transmission limit the effectiveness of autografts and allografts, respectively. Alternatively, use of decellularized bone matrices eliminates donor site morbidity and minimizes the risk to the patient from disease and immune response. However, the use of decellularized bone is still dependent on the harvesting and shaping of bone, as well as the ability to completely denude the specimen of native cells. Finally, the use of polymer scaffolds eliminates the need for organic tissue harvesting and its accompanying limitations. Polymers exhibit the ability to engineer complex geometries with tailorable physical properties. Unfortunately, many polymers display limited usefulness due to issues including incompatible mechanical properties, degradation rates, internal porosities and geometries, and the release of harmful degradation products in vivo.

Previous studies have confirmed the presence of strongly bound citrate-rich molecules that serve to stabilize the apatite nanocrystals within natural bone. The studding of apatite crystals with these citrate molecules has been identified as a critical mechanism regulating the size of the nanocrystals to a favorable thickness of three nanometers. The regulation of apatite nanostructure and the formation of apatitic calcium phosphate crystals imparts natural bone with its mechanical properties, and citrate is now thought to be a critical component in bone metabolism. Citrate based, biodegradable elastomers have been previously developed, displaying excellent in vitro and in vivo biocompatibility; however, these materials display insufficient mechanical properties in hydrated conditions, rapid degradation, and minimal osteogenic capability. The rich carboxylic acid groups of these materials display the ability to chelate with calcium-containing hydroxyapatite, facilitating polymer/hydroxyapatite interactions that are similar to the natural interaction and formation of citrate bound apatite nanocrystal in natural bone. As a result, these polymer/hydroxyapatite composites display improved mechanical properties, degradation, and bioactivity; however, compositing with hydroxyapatite and other inorganic filler materials results in materials that still do not fully match the mechanics of native bone and suffer from lengthy degradation times.

Thus, there is a clear need for materials that may be used as bone grafts or as other tissue engineering materials that show improved mechanical properties, degradation rate and bioactivity while still maintaining biodegradability. The present disclosure addresses this as well as other needs.

### SUMMARY

The present disclosure provides compositions useful as tissue engineering materials. More particularly, the present disclosure provides xylitol-doped citrate polymer compositions which may find use, for example, as bone graft materials. Methods of use and methods of making these materials are also provided.

In one aspect, a composition is provided comprising a polymer or oligomer formed from one or more monomers of Formula (A1), one or more monomers independently selected from Formula (B1) and Formula (B2), and one or more monomers of Formula (C1):

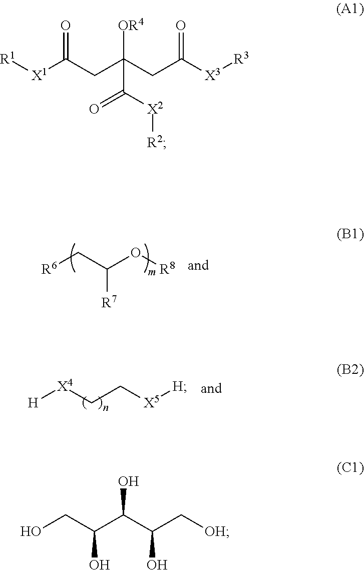

wherein:

X¹, X², and X³ are each independently —O— or —NH—; X⁴ and X⁵ are independently —O— or —NH;

R¹, R², and R³ are each independently —H, C₁-C₂₂ alkyl, C₂-C₂₂ alkenyl, or M⁺;

R⁴ is H or M⁺;

R⁶ is —H, —NH, —OH, —OCH₃, —OCH₂CH₃; —CH₃, or —CH₂CH₃; R⁷ is —H, C₁-C₂₃ alkyl, or C₂-C₂₃ alkenyl; R⁸ is —H, C₁-C₂₃ alkyl, C₂-C₂₃ alkenyl, —CH₂CH₂OH, or —CH₂CH₂NH₂;

n and m are independently integers ranging from 1 to 2000; and

M⁺ is a cation.

In some embodiments, X¹, X², and X³ are each —O—. In some embodiments, R⁴ is —H. In some embodiments, the one or more monomers of Formula (A1) comprise citric acid or a citrate. In some embodiments, the one or more monomers of Formula (B1) are selected from poly(ethylene glycol) and poly(propylene glycol). In some embodiments, the one or more monomers of Formula (B2) are selected from 1,2-ethylene glycol, 1,3-propanediol, 1,4-butanediol, 1,5-pentanediol, 1,6-hexanediol, 1,8-octanediol, 1,10-decanediol, and 1,12-dodecanediol.

In some embodiments, the one or more monomers independently selected from Formula (B1) and Formula (B2) and the one or more monomers of Formula (C2) are present in a molar ratio ranging from about 20:1 to about 1:20.

In some embodiments, the polymer or oligomer is further formed from one or more monomers of Formula (D1):

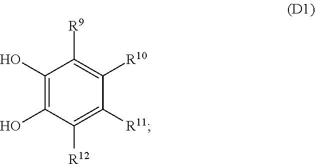

wherein:

R⁹, R¹⁰, R¹¹, and R¹² are each independently selected from —H, —OH, —CH₂(CH₂)ₓNH₂, —CH₂(CHR¹³)NH₂, —CH₂(CH₂)ₓOH, —CH₂(CHR¹³)OH, and —CH₂(CH₂)ₓCOOH:

R¹³ is —COOH or —(CH₂)_{y}COOH; and

x and y are independently an integer ranging from 1 to 10.

In some embodiments, the one or more monomers of Formula (D1) are selected from dopamine, L-DOPA, D-DOPA, gallic acid, caffeic acid, 3,4-dihydroxyhydrocinnamic acid, and tannic acid.

In some embodiments, the polymer or oligomer is further formed from one or more monomers independently selected from Formula (E1), Formula (E2), Formula (E3), and Formula (E4):

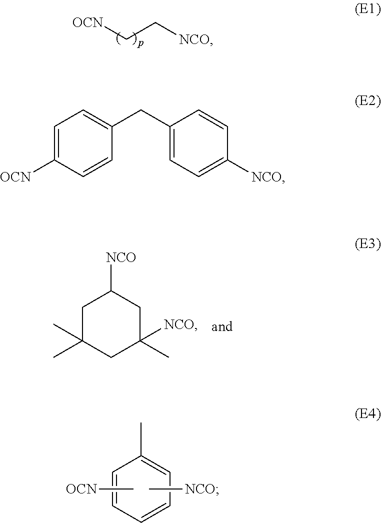

wherein p is an integer ranging from 1 to 20.

In some embodiments, the polymer or oligomer is further formed from one or more monomers independently selected from Formula (F1) and Formula (F2):

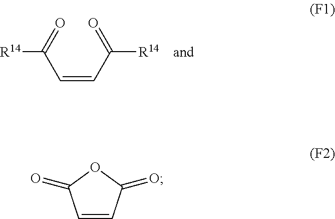

wherein R¹⁴ is selected from —OH, —OCH₃, —OCH₂CH₃, and —Cl.

In some embodiments, the polymer or oligomer is further formed from one or more monomers independently selected from Formula (G1):

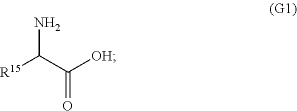

wherein R¹⁵ is an amino acid side chain.

In some embodiments, the polymer or oligomer is further formed from one or more monomers independently selected from Formula (H1), Formula (H2), and Formula (H3):

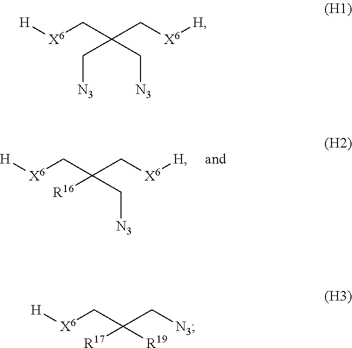

wherein:

X⁶ is independently selected at each occurrence from —O— or —NH—;

R¹⁶ is —CH₃ or —CH₂CH₃; and

R¹⁷ and R¹⁸ are each independently —CH₂N₃, —CH₃, or —CH₂CH₃.

In some embodiments, the polymer or oligomer is further formed from one or more monomers independently selected from Formula (I1), Formula (I2), Formula (I3), Formula (I4), Formula (I5), and Formula (I6):

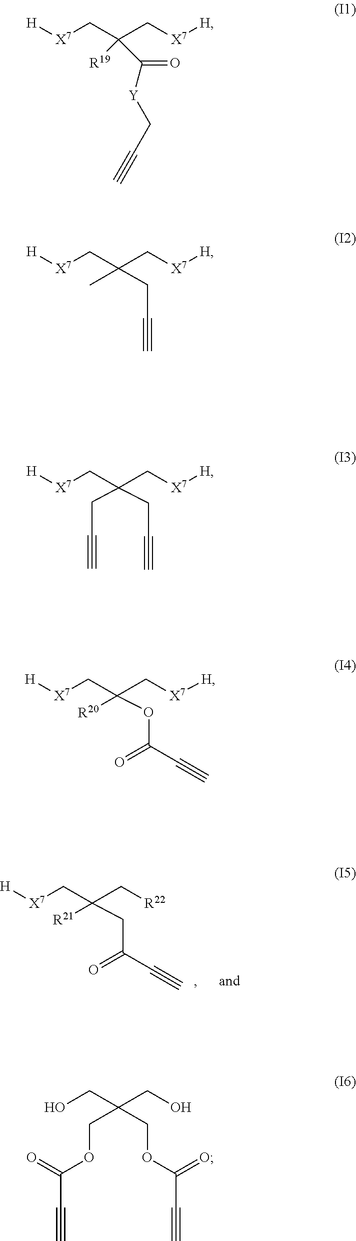

wherein:

X⁷ and Y are independently —O— or —NH—;

R¹⁹ and R²⁰ are each independently —CH₃ or —CH₂CH₃;

R²¹ is —OC(O)CCH, —CH₃, or —CH₂CH₃; and

R²² is —CH₃, —OH, or —NH₂.

In some embodiments, the polymer or oligomer is thermally crosslinked. In some embodiments, the polymer or oligomer has a cross-linking density ranging from about 600 to about 70,000 mol/m³.

In some embodiments, the composition has a tensile strength of about 1 MPa to about 120 MPa in a dry state. In some embodiments, the composition has a tensile modulus of about 1 mPA to about 3.5 GPa in a dry state. In some embodiments, the composition is luminescent.

In some embodiments, the composition further comprises an inorganic material. In some embodiments, the inorganic material is a particulate inorganic material. In some embodiments, the inorganic material is selected from hydroxyapatite, tricalcium phosphate, biphasic calcium phosphate, bioglass, ceramic, magnesium powder, pearl powder, magnesium alloy, and decellularized bone tissue particles. In such embodiments, the composition has a compressive strength ranging from about 250 MPa to about 350 MPa. In such embodiments, the composition has a compressive modulus ranging from about 100 KPa to about 1.8 GPa. In such embodiments, the composition displays room-temperature phosphorescence.

In some embodiments, the composition further comprises an antioxidant, pharmaceutically active agent, biomolecule, or cell.

In some embodiments, the composition is configured to degrade in less than 4 months.

In another aspect, a method of promoting and/or accelerating bone regeneration is provided comprising delivering a composition described herein to a bone site. In some embodiments, the composition is delivered before and/or during a proliferation stage of osteogenesis at the bone site. In some embodiments, the method further comprises delivering stem cells to the bone site. In some embodiments, the bone site is an intramembranous ossification site. In some embodiments, the bone site is an endochondral ossification site.

In another aspect, a method of preparing a composition is provided comprising:

polymerizing a polymerizable composition to form a polymer composition, the polymerizable composition comprising one or more monomers of Formula (A1), one or more monomers independently selected from Formula (B1) and Formula (B2), and one or more monomers of Formula (C1):

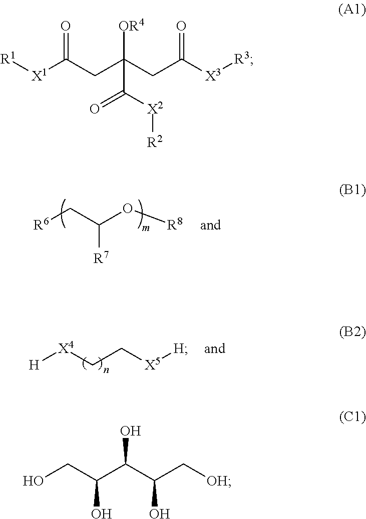

Wherein all variables are as defined herein.

In another aspect, kit for promoting and/or accelerating bone regeneration comprising a composition described herein.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a schematic showing the synthesis of representative xylitol doped poly(octamethylene citrate).

FIG. 2 shows the density of representative polymers of the disclosure as synthesized in the examples. The data demonstrates an increase in density with increased xylitol content within the polymer.

FIG. 3 shows the measured molecular weight between crosslinks in representative polymers of the disclosure as synthesized in the examples. Polymers containing xylitol were found to have a highly crosslinked structure as compared to conventional POC, leading to enhanced mechanical properties.

FIG. 4 shows the Fourier-transform infrared spectrogram for representative polymers of the disclosure as synthesized in the examples. An increased —OH signal was found with increased xylitol content, indicating the formation of hydrogen bonding between polymer chains which further reinforces polymer mechanics.

FIG. 5 are x-ray diffraction spectra for representative polymers of the disclosure as synthesized in the examples. The spectra depict a lack of crystallinity of the polymers induced by increase xylitol content.

FIGS. 6A, 6B, 6C, 6D, 6E, 6F, and 6G show tensile film mechanics for films formed from representative polymers of the present disclosure as described in the examples. These measurements demonstrate the tunability of film mechanics in a manner that is capable of matching a range of biological tissues such as skin, nerve, bone, etc.

FIGS. 7A and 7B show the measured external contact angle for representative polymers of the present disclosure as described in the examples. These data show the hydrophilicity of the representative materials.

FIG. 8. Provides data showing enhanced fluorescence of the representative polymers with increasing xylitol content.

FIGS. 9A, 9B, 9C, 9D, 9E, 9F, and 9G show fluorescence emission spectra for representative polymers of the present disclosure. These spectra show that the disclosed compositions are capable of imaging and light delivery in vivo.

FIG. 10 shows measurements of compressive stress for representative compositions of the disclosure further comprising 60 weight percent hydroxyapatitite (HA). These data demonstrate uniform stress on the compositions regardless of xylitol content.

FIG. 11 shows measurements of compressive modulus for representative formulations of the disclosure further comprising 60 weight percent hydroxyapatite. These measurements are significantly equivalent compared to composites lacking xylitol as a monomer component.

FIG. 12 shows measurements of compressive strain for representative compositions further comprising 60 weight percent hydroxyapatite (HA).

FIG. 13 shows the weight percentage of swelling for representative compositions of the present disclosure. The data show that composites containing xylitol swell at the same rate as composites lacking xylitol despite the increased hydrophilic character of said monomer component.

FIG. 14 shows the percent degradative loss of representative compositions over time. Degradation was found to be tunable from 5% to 40% (i.e., complete degradation of the polymer component) over a 16-week period. When viewed in combination with the associated mechanical data for the representative polymers, these data demonstrate wide tunability of composition degradation without any negative impact on mechanics of the composition.

FIG. 15 shows measurements of pH versus time for representative compositions of the disclosure. These data show a return to ˜7.4 pH (physiological) within one week. Therefore, the compositions of the present disclosure are capable of replicating a desired pH profile for the bone environment.

FIGS. 16A and 16B show fluorescence and room temperature phosphorescence, respectively, for compositions of the disclosure containing hydroxyapatite (POCX6/50HA). These demonstrate that the disclosed compositions may be used with multiple imaging modalities. In particular, phosphorescence may be preferred for imaging in vivo to avoid the autofluorescence of biological tissue through the intrinsic delayed emission of phosphorescence versus fluorescence.

FIGS. 17A, 17B, and 17C show in vitro cytotoxicity evaluation against MG63 cells of the degradation products for disclosed compositions as described in the examples as well as the cytotoxicity of leachable components and degradation products for such compositions further comprising hydroxyapatite (CXBE/50HA).

FIG. 18 shows imaging demonstrating cranial bone regeneration resulting from the disclosed compositions (POC-X6/50HA), showing bone regeneration similar to clinically utilized PLGA/35HA materials.

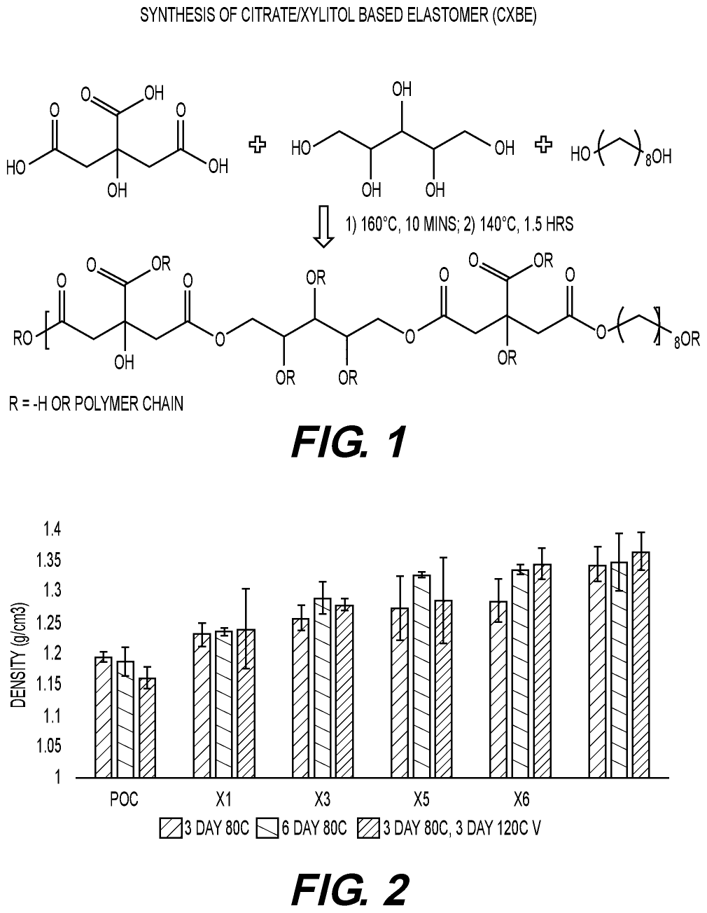

Like reference symbols in the various drawings indicate like elements.

### DETAILED DESCRIPTION

The present invention can be understood more readily by reference to the following detailed description, examples, drawings, and claims, and their previous and following description. However, before the present compositions, systems, and/or methods are disclosed and described, it is to be understood that this invention is not limited to the specific or exemplary aspects of compositions, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

The present disclosure is directed to compositions containing citrate polymers doped with xylitol along with their methods of use as tissue engineering materials, for example particularly as bone grafts. Xylitol is an FDA approved sugar alcohol that is currently used as an alternative sweetener as well as a cavity-preventing dental rinse. Xylitol contains five hydroxyl groups capable of reacting with the carboxyl group (or derivatives thereof) of citric acid or citrate derivatives. The presence of these hydroxyl groups not only allows xylitol to be incorporated into citrate-containing polymers via esterification during polymerization, but the large number of said groups also increases the number of chemical crosslinks formed. These additional crosslinks improve the mechanical strength of the polymer, particularly the modulus. In addition, the large number of hydroxyl groups found within the xylitol monomers are capable of ionic binding with calcium, either within hydroxyapatite or deposited from an outside source. This binding improves the interface between hydroxyapatite and the polymer within compositions and increases the amount of calcium and subsequent mineral deposition in the composite surface. Previous studies conducted on rats demonstrated that oral administration of xylitol increased femur mineral density as a result of increased calcium bioavailability. Further, studies have also shown significant antibacterial and antioxidant activity of xylitol. Compared to the polyols used previously in citrate-based polymers, xylitol is more biocompatible and has increased hydrophilicity, which increases the water uptake into the polymer and/or composites and increases the rate of hydrolysis. The compositions of the present disclosure show increased mechanical properties that exceed that of native bone while showing modulated degradation rates from approximately 1 year to 4 months. Therefore, the presently disclosed compositions are a system where high mechanical strength is maintained independent of to biodegradation rate.

### Definitions

As used herein, the terms “optional” or “optionally” means that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable sub-combination.

As used in the description and the appended claims, the singular forms “a,” “an,” and “the” include plural referents unless the context clearly dictates otherwise. Thus, for example, a reference to “a functional group” includes two or more such functional groups, reference to “a composition” includes two or more such compositions and the like.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification, and in the claims which follow, reference will be made to a number of terms that shall be defined herein.

For the terms “for example” and “such as,” and grammatical equivalences thereof, the phrase “and without limitation” is understood to follow unless explicitly stated otherwise.

As used herein, the term “substituted” means that a hydrogen atom is removed and replaced by a substituent. It is contemplated to include all permissible substituents of organic compounds. As used herein, the phrase “optionally substituted” means unsubstituted or substituted. It is to be understood that substitution at a given atom is limited by valency. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with a permitted valence of the substituted atom and the substituent and that the substitution results in a stable compound, e.g., a compound that does not spontaneously undergo transformation such as by rearrangement, cyclization, elimination, etc. In still further aspects, it is understood that when the disclosure describes a group being substituted, it means that the group is substituted with one or more (i.e., 1, 2, 3, 4, or 5) groups as allowed by valence selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol, as described below.

The term “aliphatic” as used herein refers to a nonaromatic hydrocarbon group and includes branched and unbranched, alkyl, alkenyl, or alkynyl groups. As used herein, the term “Cₙ-Cₘ alkyl,” employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, heptyl, octyl, nonyl, decyl, dodecyl, tetradecyl, hexadecyl, eicosyl, tetracosyl, and the like. The alkyl group can also be substituted or unsubstituted. Throughout the specification, the term “alkyl” is generally used to refer to both unsubstituted alkyl groups and substituted alkyl groups; however, substituted alkyl groups are also specifically referred to herein by identifying the specific substituent(s) on the alkyl group. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

As used herein, “Cₙ-Cₘ alkenyl” refers to an alkyl group having one or more double carbon-carbon bonds and having n to m carbons. Examples of alkenyl groups include, but are not limited to, ethenyl, n-propenyl, isopropenyl, n-butenyl, seobutenyl, and the like. In various aspects, the alkenyl moiety contains 2 to 6, 2 to 4, or 2 to 3 carbon atoms. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, cyano, silyl, sulfo-oxo, sulfonyl, sulfone, sulfoxide, thiol, or phosphonyl, as described below.

The terms “amine” or “amino” as used herein are represented by the formula —NR^{x}R^{y}, where R^{x} and R^{y} can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NR^{x}R^{y}.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)R^{z} or —C(O)OR^{z}, where R^{z} can be an alkyl, halogenated alkyl, alkenyl, alkynyl, aryl, heteroaryl, cycloalkyl, cycloalkenyl, heterocycloalkyl, or heterocycloalkenyl group described above.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within the second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be to further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the endpoints of the range unless expressly stated otherwise. For example, a range of “between 5 and 10,” “from 5 to 10,” or “5-10” should generally be considered to include the endpoints 5 and 10. Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

As used herein, the term “composition” is intended to encompass a product comprising the specified ingredients in the specified amounts, as well as any product which results, directly or indirectly, from a combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

It will be understood that, although the terms “first,” “second,” etc., may be used herein to describe various elements, components, regions, layers and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the terms “substantially identical reference composition” refers to a reference composition comprising substantially identical components in the absence of an inventive component. In another exemplary aspect, the term “substantially” in, for example, the context “substantially identical reference composition” refers to a reference composition comprising substantially identical components and wherein an inventive component is substituted with a component common in the art.

While aspects of the present invention can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Compositions

In one aspect, a composition is provided comprising a polymer or oligomer formed from one or more monomers of Formula (A1), one or more monomers independently selected from Formula (B1) and Formula (B2), and one or more monomers of Formula (C1):

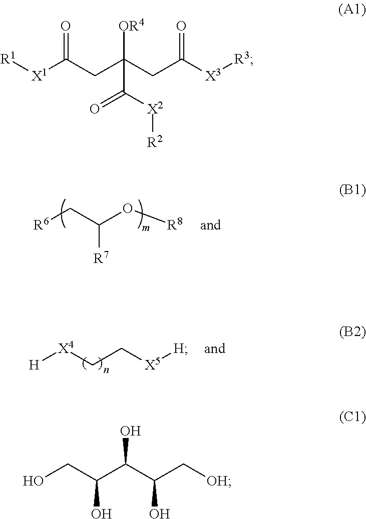

wherein:

X¹, X², and X³ are each independently —O— or —NH—;

X⁴ and X⁵ are independently —O— or —NH;

R¹, R², and R³ are each independently —H, C₁-C₂₂ alkyl, C₂-C₂₂ alkenyl, or M⁺;

R⁴ is H or M⁺;

R⁶ is —H, —NH, —OH, —OCH₃, —OCH₂CH₃; —CH₃, or —CH₂CH₃;

R⁷ is —H, C₁-C₂₃ alkyl, or C₂-C₂₃ alkenyl;

R⁸ is —H, C₁-C₂₃ alkyl, C₂-C₂₃ alkenyl, —CH₂CH₂OH, or —CH₂CH₂NH₂;

n and m are independently integers ranging from 1 to 2000; and

M⁺ is a cation.

In some embodiments, X¹ is —O—. In some embodiments, X² is —O—. In some embodiments, X³ is —O—. In some embodiments, X¹, X², and X³ are each —O—.

In some embodiments, X⁴ is —O—. In some embodiments, X⁴ is —NH—. In some embodiments, X⁵ is —O—. In some embodiments, X⁵ is —NH—. In some embodiments, X⁴ and X⁵ are each —O—. In some embodiments, X⁴ and X⁵ are each —NH—. In some embodiments, one of X⁴ and X⁵ is —O— and the other of X⁴ and X⁵ is —NH—.

In some embodiments, R¹, R², and R³ are each independently —H, —CH₃, or —CH₂CH₃.

In some embodiments, R¹, R² and R³ are each independently —H or M⁺.

In some embodiments, R⁴ is —H.

In some embodiments, R⁴ is M⁺.

In some embodiments, M⁺ is independently at each occurrence Na⁺ or K⁺.

In some embodiments, R⁶ is —OH.

In some embodiments, R⁷ is —H. In some embodiments, R⁷ is —CH₃.

In some embodiments, R⁸ is —H.

In some embodiments, n and m can independently be an integer from 1 to 2000, including exemplary values of 1 to 100, or 1 to 250, or 1 to 500, or 1 to 750 or 1 to 1000, or 1 to 1250, or 1-1500, or 1 to 1750. In yet other aspects, n and m can independently be an integer between 1 and 20, including exemplary values of 2, 3, 4, 5, 6, 7, 8, 9, 10, 11, 12, 13, 14, 15, 16, 17, 18, and 19.

In some embodiments, the one or more monomers of Formula A1 can comprise an alkoxylated, alkenoxylated, or non-alkoxylated and non-alkenoxylated citric acid, citrate, or ester or amide of citric acid.

In some embodiments, the one or more monomers of Formula B1 are selected from poly(ethylene glycol) (PEG) and poly(propylene glycol) (PPG) having terminal hydroxyl or amine groups. Any such PEG or PPG not inconsistent with the objected of the present disclosure may be used. In some embodiments, for example, a PEG or PPG having a weight average molecular weight between about 100 and about 5000 or between about 200 and about 1000 or between 200 and about 100,000 may be used.

In some embodiments, the one or more monomers of Formula B2 may comprise C₂-C₂₀, C₂-C₁₂, or C₂-C₆ aliphatic alkane diols or diamines. For instance, the one or more monomers of Formula B2 may comprise 1,4-butanediol, 1,4-butanediamine, 1,6-hexanediol, 1,6-hexanediamine, 1,8-octanediol, 1,8-octanediamine, 1,10-decanediol, 1,10-decanediamine, 1,12-dodecanediol, 1,12-dodecanediamine, 1,16-hexadecanediol, 1,16-hexadecanediamine, 1,20-icosanediol, or 1,20-icosanediamine. In alternative embodiments, the one or more monomers of Formula B2 may be replaced by a branched alkanediol/diamine, alkenediol/diamine, or an aromatic diol/diamine.

In some embodiments, the polymer may be formed from a molar ratio of the one or more monomers of Formula (A1) to the one or more monomers of Formula (B1), Formula B2), and Formula (C1) [A1:(B1+B2+C1)] ranging from about 3:1 to about 1:3, for example about 3:1, about 2.5:1, about 2:1, about 1.5:1, about 1:1, about 1:1.5, about 1:2, about 1:2.5, or about 1:3.

In some embodiments, the polymer or oligomer may further be formed from one or more monomers comprising a catechol-containing species. The catechol containing species can comprise any catechol-containing species not inconsistent with the objects of the present disclosure. In some cases, a catechol-containing species comprises at least one moiety that can form an ester or amide bond with another chemical species used to form a polymer in embodiments were the monomers are reacted. For example, in some embodiments, a catechol-containing species comprises an alcohol moiety, an amine moiety, a carboxylic acid moiety, or combinations thereof. Further, in some embodiments, a catechol-containing species comprises a hydroxyl moiety that is not part of the catechol moiety. In some embodiments, a catechol-containing species comprises dopamine. In other embodiments, a catechol-containing species comprises L-3,4-dihydroxyphenylalanine (L-DOPA) or D-3,4-dihydroxyphenylalanine (D-DOPA). In still other embodiments, a catechol-containing species comprises gallic acid or caffeic acid. In some embodiments, a catechol-containing species comprises 3,4-dihydroxycinnamic acid. Additionally, a catechol-containing species may also comprise a naturally-occurring species or a derivative thereof, such as tannic acid or a tannin. Moreover, in some embodiments, a catechol-containing species is coupled to the backbone of the polymer or oligomer through an amide bond. In other embodiments, a catechol-containing species is coupled to the backbone of the polymer or oligomer through an ester bond. Further examples of catechol-containing species can be found in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers of Formula (D1):

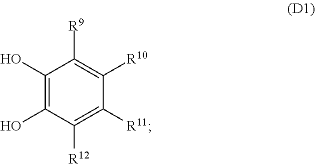

wherein:

R⁹, R¹⁰, R¹¹ and R¹² are each independently selected from —H, —OH, —CH₂(CH₂)ₓNH₂, —CH₂ (CHR¹³)NH₂, —CH₂(CH₂)ₓOH, —CH₂(CHR¹³)OH, and —CH₂(CH₂)ₓCOOH:

R¹³ is —COOH or —(CH₂)_{y}COOH; and

x and y are independently an integer ranging from 1 to 10.

In some embodiments, the one or more monomers of Formula (D1) are selected from dopamine, L-DOPA, D-DOPA, gallic acid, caffeic acid, 3,4-dihydroxyhydrocinnamic acid, and tannic acid.

In some embodiments, the polymer or oligomer may further be formed from one or more monomers comprising a diisocyanate. In some embodiments, an isocyanate comprises an alkane diisocyanate having four to twenty carbon atoms. An isocyanate described herein may also include a monocarboxylic acid moiety. Further examples of various isocyanates which can be used are described in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers independently selected from Formula (E1), Formula (E2), Formula (E3), to and Formula (E4):

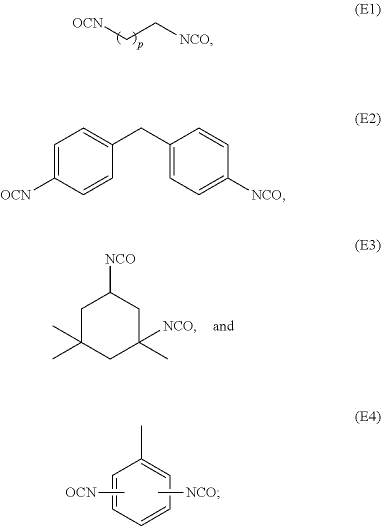

wherein p is an integer ranging from 1 to 20.

In some embodiments, the polymer or oligomer may further be formed from one or more monomers comprising a polycarboxylic acid, such as a dicarboxylic acid, or a functional equivalent of a polycarboxylic acid, such as a cyclic anhydride or an acid chloride of a polycarboxylic acid. In some embodiments, the polycarboxylic acid or functional equivalent thereof can be saturated or unsaturated. In some embodiments, for example, the polycarboxylic acid or functional equivalent thereof comprises maleic acid, maleic anhydride, fumaric acid, or fumaryl chloride. In some embodiments, a vinyl-containing polycarboxylic acid or functional equivalent thereof may also be used, such as allylmalonic acid, allylmalonic chloride, itaconic acid, or itaconic chloride. Further, in some embodiments, the polycarboxylic acid or functional equivalent thereof can be at least partially replaced with an olefin-containing monomer that may or may not be a polycarboxylic acid. In some embodiments, for instance, an olefin-containing monomer comprises an unsaturated polyol such as a vinyl-containing diol. Further examples can be found in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers independently selected from Formula (F1) or Formula (F2):

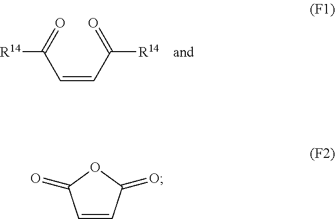

wherein R¹⁴ is selected from —OH, —OCH₃, —OCH₂CH₃, or —Cl.

In some embodiments, the polymer or oligomer may further be formed from one or more monomers comprising an amino acid, such as an alpha-amino acid. An alpha-amino acid of a polymer described herein, in some embodiments, comprises an L-amino acid, a D-amino acid, or a D,L-amino acid. In some embodiments, an alpha-amino acid comprises alanine, arginine, asparagine, aspartic acid, cysteine, glycine, glutamine, glutamic acid, histidine, isoleucine, leucine, lysine, methionine, proline, phenylalanine, swine, threonine, tyrosine, tryptophan, valine, or a combination thereof. Further, in some embodiments, an alpha-amino acid comprises an alkyl-substituted alpha-amino acid, such as a methyl-substituted amino acid derived from any of the 22 “standard” or proteinogenic amino acids, such as methyl serine.

In some embodiments, the polymer or oligomer may further be formed from one or more monomers independently selected from Formula (G1):

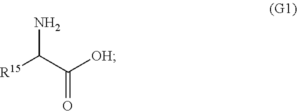

wherein R¹⁵ is an amino acid side chain.

In some embodiments, the polymer or oligomer may further be formed from one or more monomers comprising one or more alkyne moieties and/or one or more azide moieties. The monomer comprising one or more alkyne and/or azide moieties used to form a polymer described herein can comprise any alkyne- and/or azide-containing chemical species not inconsistent with the objectives of the present disclosure. Additional examples of monomers containing alkyne and/or azide moieties can be found in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers independently selected from Formula (H1), Formula (H2), and Formula (H3):

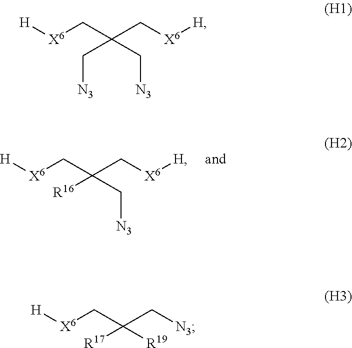

wherein:

X⁶ is independently selected at each occurrence from —O— or —NH—;

R¹⁶ is —CH₃ or —CH₂CH₃; and

R¹⁷ and R¹⁸ are each independently —CH₂N₃, —CH₃, or —CH₂CH₃.

In some embodiments, the polymer or oligomer may further be formed from one or more monomers independently selected from Formula (I1), Formula (I2), Formula (I3), Formula (I4), Formula (I5), and Formula (I6):

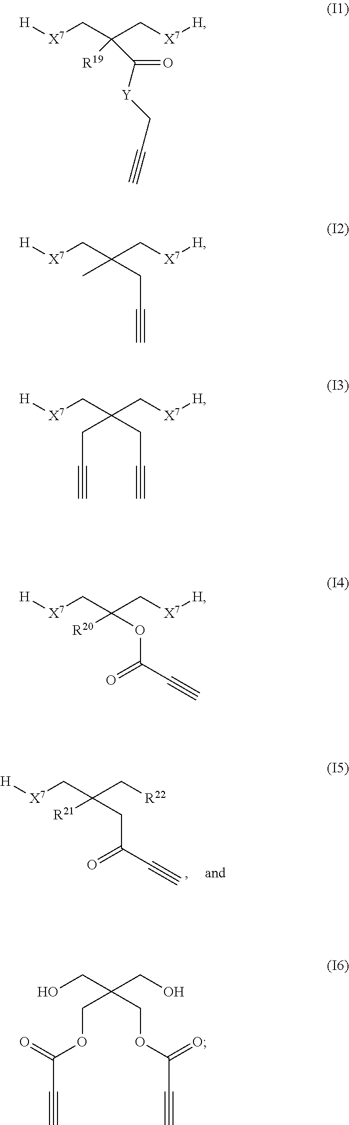

wherein:

X⁷ and Y are independently —O— or —NH—;

R¹⁹ and R²⁰ are each independently —CH₃ or —CH₂CH₃;

R^{2′} is —OC(O)CCH, —CH₃, or —CH₂CH₃; and

R²² is —CH₃, —OH, or —NH₂.

In some embodiments, a monomer described herein can be functionalized with a bioactive species. Moreover, said monomer can comprise one or more alkyne and/or azide moieties. For example, in some embodiments, a polymer or oligomer described herein is formed from one or more monomers containing a peptide, polypeptide, nucleic acid, or polysaccharide, wherein the peptide, polypeptide, nucleic acid, or polysaccharide is functionalized with one or more alkyne and/or azide moieties. In some embodiments, the bioactive species described herein is a growth factor or signaling molecule. Further, the peptide can comprise a dipeptide, tripeptide, tetrapeptide, or a longer peptide.

In some embodiments, the stoichiometric ratio of carboxylic acid groups or derivatives thereof to hydroxyl groups within the monomers used to form the polymer or oligomer is about 1:1. In some embodiments, the stoichiometric ratio of carboxylic acid groups or derivatives thereof to hydroxyl groups within the monomers used to form the polymer or oligomer is less than about 1:1. If the stoichiometric ratio is less than about 1:1, the polymer or oligomer may show defined regions of hydrogen bonding.

A composition described herein, in some cases, is a condensation polymerization reaction product of the identified species. Thus, in some embodiments, at least two of the identified species are co-monomers for the formation of a copolymer. In some such embodiments, the reaction product forms an alternating copolymer or a statistical copolymer of the co-monomers. Additionally, as described further herein, species described herein may also form pendant groups or side chains of a copolymers.

Additionally, in some embodiments, a composition comprising a polymer described herein can further comprise a crosslinker. Any crosslinker not inconsistent with the objectives of the present disclosure may be used. In some cases, for example, a crosslinker comprises one or more olefins or olefinic moieties that can be used to crosslink polymers containing ethylenically unsaturated moieties. In some embodiments, a crosslinker comprises an acrylate or polyacrylate, including a diacrylate. In other embodiments, a crosslinker comprises one or more of 1,3-butanediol diacrylate, 1,6-hexanediol diacrylate, glycerol 1,3-diglyerolate diacrylate, d(ethylene glycol) diacrylate, poly(ethylene glycol) diacrylate, poly(propylene glycol) diacrylate, and propylene glycol glycerolate diacrylate. In still other embodiments, a crosslinker comprises a nucleic acid, including DNA or RNA. In still other instances, a crosslinker comprises a “click chemistry” reagent, such as an azide or an alkyne. In some embodiments, a crosslinker comprises an ionic crosslinker. For instance, in some embodiments, a polymer is crosslinked with a multivalent metal ion, such as a transition metal ion. In some embodiments, a multivalent metal ion used as a crosslinker of the polymer comprises one or more of Fe, Ni, Cu, Zn, or Al, including in the +2 or +3 state.

In addition, a crosslinker described herein can be present in a composition in any amount not inconsistent with the objective of the present disclosure. For example, in some embodiments, a crosslinker is present in a composition in an amount between about 5 weight percent and about 50 weight percent, between about 5 weight percent and about 40 weight percent, between about 5 weight percent and about 30 weight percent, between about 10 weight percent and about 40 weight percent, between about 10 weight percent and about 30 weight percent, or between about 20 weight percent and about 40 weight percent, based on the total weight of the composition.

Thus, in some embodiments, the composition described herein comprises a polymer described herein that is crosslinked to from a polymer network. In some embodiments, the polymer network comprises a hydrogel. A hydrogel, in some cases, comprises an aqueous continuous phase and polymeric disperse or discontinuous phase. Further in some embodiments, the crosslinked polymer network described herein is not water soluble.

Such a polymer network can have a high cross-linking density. “Cross-linking density”, for reference purposes herein, can refer to the number of cross-links between polymer backbones or the molecular weight between cross-linking sites. Cross-links may include, for example, ester bonds formed by the esterification or reaction of one or more pendant carboxyl or carboxylic acid groups with one or more pendant hydroxyl groups of adjacent polymer backbones. In some embodiments, a polymer network described herein has a cross-linking density of at least about 500, at least about 1000, at least about 5000, at least about 7000, at least about 10,000, at least about 20,000, or at least about 30,000 mol/m³. In some embodiments, the cross-linking density is between about 600 and about 70,000, or between about 10,000 and about 70,000 mol/m³.

In some embodiments, the compositions described herein show decreased molecular weight and increasing crosslink density as compared to a substantially identical reference composition not formed from a monomer of Formula (C1).

In some embodiments, the compositions described herein show increased hydrophilicity as compared to a substantially identical reference composition not formed from a monomer of Formula (C1).

In some embodiments, the compositions described herein show increased fluorescence as compared to a substantially identical reference composition not formed from a monomer of Formula (C1).

In some embodiments, the compositions described herein can exhibit a tensile strength of about 1 MPa to about 120 MPa in a dry state as measured according to ASTM Standard D412A, for example of about 2 MPa, 10 MPa, 20 MPa, 30 MPa, 40 MPa, 50 MPa, 60 MPa, 70 MPa, 80 MPa, 90 MPa, or 100 MPa.

In some embodiments, the compositions described herein can exhibit a tensile modulus of about 1 MPa to about 3.5 GPa in a dry state as measured according to ASTM Standard D412A, for example about 1 MPa, about 10 MPa, about 50 MPa, about 100 MPa, about 250 MPa, about 500 MPa, about 750 MPa, about 1 GPa, about 1.5 GPa, about 2 GPa, about 2.5 GPa, about 3 GPa, or about 3.5 GPa.

The compositions described herein can be useful for promoting and/or accelerating bone regeneration, including bone growth, bone healing, and/or bone repair as further described herein. It should be understood that one or more compositions described herein can be used in one or more methods of promoting and/or accelerating bone regeneration described herein, including for bone growth, bone healing, and/or bone repair.

In some embodiments, the compositions described herein useful for promoting bone growth can comprise a graft or scaffold. A “graft” or “scaffold”, for reference purposes herein, can refer to any structure usable as a platform or implant for the replacement of missing bone or for promotion of growth of new bone. Moreover, as utilized herein, a “graft” or “scaffold” may be synonymous. For example, a graft or scaffold composition described herein can be used in the repair of a bone defect, the replacement of missing or removed bone, or for the promotion of new bone growth, as in the case of a bone fusion procedure. Further, it is to be understood that grafts or scaffolds consistent with compositions and methods described herein can have any structure or be formed in any shape, configuration, or orientation not inconsistent with the objected of the present disclosure. For example, in some embodiments, a graft or scaffold can be shaped, configured, or oriented in such a manner as to correspond to a defect or bone growth site to be repaired. For example, in some embodiments, a graft or scaffold utilized in the repair of a bone defect, such as a cranial defect of condyle defect, may be formed, molded, or resized to a size and/or shape corresponding to the defect. In certain other cases, such as in a bone fusion procedure, a graft or scaffold in composition and methods described herein can have a shape, configuration, orientation, or dimensions adapted to traverse a gap between the bones to be fused and/or to reinforce a bone growth site. In this manner, particular shapes, sizes, orientations and/or configurations of grafts or scaffolds described herein are not intended to be limited to a particular set or subset of modalities on, within, or adjacent to a bone growth site. A “bone site”, as referenced herein, can be any area in which bone regeneration, bone ossification, bone growth, or bone repair may be desired. In certain non-limiting examples, a bone site can comprise or include a bone defect, a site in which bone has been removed or degraded, and/or a site of desired new bone growth or regeneration, as in the case of a spine or other bone fusion.

Various components of compositions which may form part or all of a graft or scaffold utilized for promoting bone regeneration have been described herein. It is to be understood that a composition according to the present disclosure can comprise any combination of components and features not inconsistent with the objectives of the present disclosure. For example, in some cases, a composition forming part or all of a graft or scaffold utilized in a composition described herein can comprise a combination, mixture, or blend of polymers described herein. Additionally, in some embodiments, such a combination, mixture, or blend can be selected to provide a graft or scaffold having any osteo-promoting property, biodegradability, mechanical property, and/or chemical functionality described herein.

Further, one or more polymers described herein can be present in a composition forming part or all of a graft or scaffold utilized in any amount not inconsistent with the objectives of the present disclosure. In some embodiments, a graft or scaffold consists or consists essentially of the one or more polymers described herein. In other instances, a graft or scaffold comprises up to about 95 weight percent, up to about 90 weight percent, up to about 80 weight percent, up to about 70 weight percent, up to about 60 weight percent, up to about 50 weight percent, up to about 40 weight percent, or up to about 30 weight percent polymer, based on the total weight of the graft or scaffold. In some embodiments, the balance of a graft or scaffold described herein can be water, an aqueous solution, and/or an inorganic material as described further below.

In some embodiments, the composition can further comprise an inorganic material. In some embodiments, the inorganic material comprises a particulate inorganic material. Any particulate inorganic material not inconsistent with the objectives of the present disclosure may be used. In some cases, the particulate inorganic material comprises one or more of hydroxyapatite, tricalcium phosphate (including alpha- and beta-tricalcium phosphate), biphasic calcium phosphate, bioglass, ceramic, magnesium powder, pearl powder, magnesium alloy, and decellularized bone tissue particle. Other particular materials may also be used.

In addition, a particular inorganic material described herein can have any particle size and/or particle shape not inconsistent with the objective of the present disclosure. In some embodiments, for instance, a particulate material has an average particle size in at least one dimension of less than about 1000 μm, less than about 800 μm, less than about 500 μm, less than about 300 μm, less than about 100 μm, less than about 50 μm, less than about 30 μm, or less than about 10 μm. In some cases, a particular material has an average particle size in at least one dimension of less than about 1 μm, less than about 500 nm, less than about 300 nm, less than about 100 nm, less than about 50 nm, or less than about 30 nm. In some instances, a particulate material has an average particle size recited herein in two dimension or three dimensions. Moreover, a particulate material can be formed of substantially spherical particles, plate-lite particles, needle-like particles, or a combination thereof. Particulate materials having other shapes may also be used.

A particular inorganic material can be present in the compositions (such as a graft or scaffold) described herein in any amount not inconsistent with the objective of the present disclosure. For example, in some cases, a composition utilized as a graft or scaffold described herein comprises up to about 30 weight percent, up to about 40 weight percent, up to about 50 weight percent, up to about 60 weight percent, or up to about 70 weight percent particular materials, based on the total weight of the composition. In some instances, a composition comprises between about 1 and about 70 weight percent, between about 10 and about 70 weight percent, between about 15 and about 60 weight percent, between about 25 and about 65 weight percent, between about 26 and about 50 weight percent, between about 30 and about 70 weight percent, or between about 50 and about 70 weight percent particulate material, based on the total weight of the composition. For example, a composition described herein may comprise up to about 65 weight percent hydroxyapatite.

In some embodiments, the compositions further comprising inorganic materials can have a compressive strength exceeding native bone. In some embodiments, such compositions can have a compressive strength as measured by ASTM Standard D695-15 of about 250 MPa to about 350 MPa, for example about 275 MPa, 300 MPa, or 325 MPa.

In some embodiments, compositions described herein further comprising inorganic materials can have a compressive modulus as measured by ASTM Standard D695-15 of about 100 KPa to about L8 GPa, for example about 100 KPa, about 10 MPa, about 50 MPa, about 100 MPa, about 250 MPa, about 500 MPa, about 750 MPa, about 1.0 GPa, about 1.2 GPa, about 1.4 GPa, about 1.6 GPa, or about 1.8 GPa.

In some embodiments, compositions described herein further comprising inorganic materials may display room temperature phosphorescence.

In another aspect, incorporation of monomer of Formula (C1) in the compositions described herein does not substantially increase swelling of the composite material.

In some embodiments, the graft or scaffold may be itself a particulate. The particulate graft or scaffold may include or contain a liquid or be substantially “dry” or free of liquid. Moreover, such a liquid that is included in (or mostly excluded from) such a to particular graft or scaffold can be any liquid not inconsistent with the objectives of the present disclosure. In some embodiments, for instance, the liquid is water or an aqueous solution or mixture, such as saline. Moreover, in some embodiments, the liquid can be a carrier liquid for introducing other species to the particulate graft or scaffold. For example, in some embodiments, the liquid comprises one or more biomolecules, bioactive materials, or other biomaterials, as described further below. In some embodiments, the liquid comprises a hyaluronate or hyaluronic acid. In other embodiments, the liquid comprises blood or plasma.

Additionally, the particulate graft or scaffold, in some embodiments, is a paste. More particularly, such a paste can include the particulate graft or scaffold and a liquid (as opposed to being a “dry” material). Such a “paste” can be a viscous or shape-stable material (at standard temperature and pressure conditions) and can have a viscosity suitable for handling or manipulation, such as scooping, with a microspatula. For example, in some embodiments, the paste has a dynamic viscosity of at least 1.0×10⁴ centipoise (cP), at least 5.0×10⁴, or at least 1.0×10⁵. In other embodiments, the paste has a viscosity between about 1.0×10⁴ cP and 1.0×10⁷ cP, between about 1.0×10⁵ cP and 1.0×10⁶ cP, or between 1.0×10⁶ cP and 1.0×10⁷ cP. The liquid component of a paste, in some embodiments, is an isotonic solution, and the paste is a biologically sterile paste. For example, in some embodiments, a paste described herein, can be formed from a salt solution, such as saline, or other biologically active solution such a sodium hyaluronate or blood. In some embodiments, the biologically active solution can comprise additional biological molecules or factors suitable to promote and/or accelerate bone regeneration. For example, the solution can comprise growth factors or signaling molecules, such as osteogenic factors. Non-limiting examples of biological factors that may be used in some embodiments described herein include osteopontin (OPN), osteocalcin (OCN), bone morphogenetic protein-2 (BMP-2), transforming growth factor β3 (TGFβ3), stromal cell-derived factor-1α (SDF-1α), erythropoietin (Epo), vascular endothelial growth factor (VEGF), insulin-like growth factor-1 (IGF-1), platelet derived growth factor (PDGF), fibroblast growth factor (BGF), nerve growth factor (NGF), neurotrophin-3 (NT-3), and glial cell-derived neurotrophic factor (GDNF). Other therapeutic proteins and chemical species may also be used.

In some embodiments, the graft or scaffold described herein is a polymer network. The polymer network can comprise any combination of polymers and/or copolymers described above. Further, in some embodiments, the polymer network comprises an inorganic material (such as a particulate inorganic material). For example, polymers as described above can be cross-linked to encapsulate or otherwise bond to the inorganic material. Cross-linking can be performed, for example, by exposing the polymer to heat and/or UV light.

In other embodiments, the composition described herein can have additional desirable properties suitable for use in methods described herein. In some embodiments, the composition is luminescent. In some cases, such luminescence is photoluminescence and can be observed by exposing the composition to suitable wavelength of light, such as light having a peak or average wavelength between 400 nm and 600 nm. Moreover, in some embodiments, the luminescence intensity of the composition, measured in arbitrary or relative units, can be used as a measure of degradation of the scaffold over time, thereby indicating biodegradability or clearance from a site, such as a bone site.

In some embodiments, the compositions described herein deliver citrate and xylitol to the site of action (such as a bone site) due to their release upon degradation of the composition. In some embodiments, release of xylitol and citrate may enhance osteogenic differentiation and tissue regeneration. In some embodiments, release of xylitol may increase osteogenic tissue regeneration by enhancing bioavailability of calcium. In some embodiments, release of xylitol exerts antioxidant and anti-inflammatory action on surrounding cells and/or tissues. In some embodiments, release of xylitol and citrate may exert an antimicrobial effect such that it prevents local or implant-associated infection.

### Methods of Preparation

Further provided are methods of preparing the compositions as described hereinabove. In one aspect, a method is provided for preparing a composition as described herein comprising polymerizing a polymerizable composition comprising:

one or monomers of Formula (A1):

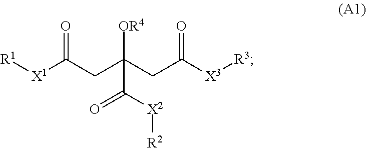

one or more monomers independently selected from Formula (B1) and Formula (B2):

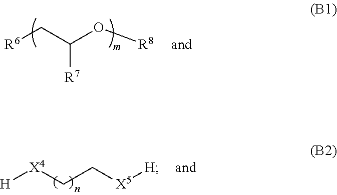

and

and one or more monomers of Formula (C1)

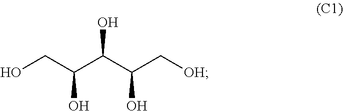

to form a polymer;

wherein:

X¹, X², and X³ are each independently —O— or —NH—;

X⁴ and X⁵ are independently —O— or —NH;

R¹, R², and R³ are each independently —H, C₁-C₂₂ alkyl, C₂-C₂₂ alkenyl, or M⁺;

R⁴ is H or M⁺;

R⁶ is —H, —NH, —OH, —OCH₃, —OCH₂CH₃; —CH₃, or —CH₂CH₃;

R⁷ is —H, C₁-C₂₃ alkyl, or C₂-C₂₃ alkenyl;

R⁸ is —H, C₁-C₂₃ alkyl, C₂-C₂₃ alkenyl, —CH₂CH₂OH, or —CH₂CH₂NH₂;

n and m are independently integers ranging from 1 to 2000; and

M⁺ is a cation.

In some embodiments, X¹ is —O—. In some embodiments, X² is —O—. In some embodiments, X³ is —O—. In some embodiments, X¹, X², and X³ are each —O—.

In some embodiments, X⁴ is —O—. In some embodiments, X⁴ is —NH—. In some embodiments, X⁵ is —O—. In some embodiments, X⁵ is —NH—. In some embodiments, X⁴ and X⁵ are each —O—. In some embodiments, X⁴ and X⁵ are each —NH—. In some embodiments, one of X⁴ and X⁵ is —O— and the other of X⁴ and X⁵ is —NH—.

In some embodiments, R¹, R², and R³ are each independently —H, —CH₃, or —CH₂CH₃.

In some embodiments, R¹, R² and R³ are each independently —H or M⁺.

In some embodiments, R⁴ is —H.

In some embodiments, R⁴ is M⁺.

In some embodiments, M⁺ is independently at each occurrence Na⁺ or K⁺.

In some embodiments, R⁶ is —OH.

In some embodiments, R⁷ is —H. In some embodiments, R⁷ is —CH₃.

In some embodiments, R⁸ is —H.

In some embodiments, n and m can independently be an integer from 1 to 2000, including exemplary values of 1 to 100, or 1 to 250, or 1 to 500, or 1 to 750 or 1 to 1000, or 1 to 1250, or 1-1500, or 1 to 1750. In yet other aspects, n and m can independently be an integer between 1 and 20, including exemplary values of 2, 3, 4, 5, 6, 7, 8, 9, 10, 11, 12, 13, 14, 15, 16, 17, 18, and 19.

In some embodiments, the one or more monomers of Formula A1 can comprise an alkoxylated, alkenoxylated, or non-alkoxylated and non-alkenoxylated citric acid, citrate, or ester or amide of citric acid.

In some embodiments, the one or more monomers of Formula B1 are selected from poly(ethylene glycol) (PEG) and poly(propylene glycol) (PPG) having terminal hydroxyl or amine groups. Any such PEG or PPG not inconsistent with the objected of the present disclosure may be used. In some embodiments, for example, a PEG or PPG having a weight average molecular weight between about 100 and about 5000 or between about 200 and about 1000 or between 200 and about 100,000 may be used.

In some embodiments, the one or more monomers of Formula B2 may comprise C₂-C₂₀, C₂-C₁₂, or C₂-C₆ aliphatic alkane diols or diamines. For instance, the one or more monomers of Formula B2 may comprise 1,4-butanediol, 1,4-butanediamine, 1,6-hexanediol, 1,6-hexanediamine, 1,8-octanediol, 1,8-octanediamine, 1,10-decanediol, 1,10-decanediamine, 1,12-dodecanediol, 1,12-dodecanediamine, 1,16-hexadecanediol, 1,16-hexadecanediamine, 1,20-icosanediol, or 1,20-icosanediamine. In alternative embodiments, the one or more monomers of Formula B2 may be replaced by a branched alkanediol/diamine, alkenediol/diamine, or an aromatic diol/diamine.

In another aspect, the method may further comprise crosslinking the polymer to provide a crosslinked polymer. The polymer may be crosslinked using any of the appropriate methods for crosslinking described herein and as would be readily apparent to those of skill in the art. In some embodiments, the polymer is crosslinked using a crosslinker. In some embodiments, crosslinking the polymer comprises thermally crosslinking the polymer.

In some embodiments, the polymer is solvent cast to form a film prior to crosslinking (such as thermal crosslinking). In other embodiments, the polymer is mixed with an inorganic material to form a homogenous mixture as described herein prior to crosslinking (such as thermal crosslinking). In some embodiments, the homogenous mixture is molded prior to crosslinking (such as thermal crosslinking).

In some embodiments, the method further comprises adding at least one biologically active agent to the formed composition.

Methods of Promoting and/or Accelerating Bone Regeneration

In another aspect, methods of promoting and/or accelerating bone regeneration are described herein. Methods described herein can use one or more compositions described herein. For example, in some embodiments, a method of promoting and/or accelerating bone regeneration comprises delivering a composition to a bone site. The composition, in some cases, comprises a biodegradable scaffold. Additionally, in some instances, a method described herein further comprises delivering stem cells to the bone site. The bone site, in some embodiments, is an intramembranous ossification site. In other embodiments, the bone site is an endochondral ossification site.

Methods of promoting and/or accelerating bone regeneration, as described herein, in some embodiments, can further comprise delivering stem cells to the bone site. For example, a graft or scaffold delivered to a bone site consistent with the methods described herein, in some embodiments, can be delivered to a bone site that is seeded with or contains a biofactor or seed cell. In some embodiments, a graft or scaffold can be seeded with a biofactor or cell such as mesenchymal stem cells (MSCs). In certain other embodiments, a graft or scaffold can be delivered to a bone site in addition to or in combination with an autologous bone graft. Biofactors or cells utilized in combination with a graft or scaffold described herein may be isolated or sourced from any host or by any means not inconsistent with the objectives of the present disclosure. For example, in some embodiment, the biofactor or cells can be harvested or isolated from the individual receiving the graft or scaffold. In certain other embodiments, the biofactor or cells can be harvested or isolated from a different individual, such as a compatible donor. In some other cases, the biofactor or cells can be grown or cultured from any individual such as the graft or scaffold recipient or another compatible individual. In certain other cases, the graft or scaffold is unseeded with a biofactor or cell upon disposition within, on, or near the bone site. Non-limiting examples of seed cells that me be used in some embodiments herein include mesenchymal stem cells (MSCs), bone marrow stromal cells (BMSCs), induced pluripotent stem (iPS) cells, endothelial progenitor cells, and hematopoietic stem cells (HSCs). Other cells may also be used. Non-limiting examples of biofactors that may be used in some embodiments described herein include bone morphogenetic protein-2 (BMP-2), transforming growth factor β3 (TGFβ3), stromal cell-derived factor-1α (SDF-1α), erythropoietin (Epo), vascular endothelial growth factor (VEGF), insulin-like growth factor-1 (IGF-1), platelet derived growth factor (PDGF), fibroblast growth factor (BGF), nerve growth factor (NGF), neurotrophin-3 (NT-3), and glial cell-derived neurotrophic factor (GDNF). Other therapeutic proteins and chemical species may also be used.

Methods of promoting and/or accelerating bone regeneration, in some embodiments, can also comprise or include additional steps. Individual steps may be carried out in any order or in any manner not inconsistent with the objectives of the present disclosure. For example, in some embodiments, methods described herein further comprise reestablishing a blood supply to the bone site and/or a biological region adjacent to the bone site. In certain cases, reestablishing a blood supply can comprise or include sealing or suturing biological tissue adjacent to the bone site. Additionally, in some cases, where blood flow has been artificially restricted at or adjacent to the bone site, such as by clamping or suction, reestablishing a blood supply can comprise or include releasing or removing the artificial restriction. Further, in some cases, a method of promoting and/or accelerating bone regeneration can comprise or include increasing one or more of osteoconduction, osteoinduction, osteogenesis, and angiogenesis within the bone site and/or a biological area adjacent to the bone site. Additionally, in some instances, methods further comprise stimulating regeneration of bone and/or soft tissue proximate to the bone site.

In some embodiments, the bone site is an intramembranous ossification site. For example, recruitment of resident mesenchymal stem cells and/or MSCs provided in methods described above can transform or differentiate into osteoblasts at the bone site. An intramembranous ossification site can be any developed or developing intramembranous bone tissue in need of bone regeneration.

In other embodiments, the bone site is an endochondral ossification site. For example, recruitment and/or proliferation of resident chondrocytes and/or differentiated MSCs provided in methods described above can further promote and/or accelerate bone regeneration at the bone site. An endochondral ossification site can be any developed or developing cartilaginous bone tissue in need of bone regeneration.

Moreover, in some embodiments, methods of promoting and/or accelerating bone regeneration described herein can comprise delivering a graft or scaffold, as described above, before and/or during an early state of osteogenic differentiation at the bone site. For example, the scaffold, in some cases, is delivered during early stages of bone regeneration, such as the proliferation stage and/or matrix maturation stage, occurring after initiation of osteogenic differentiation and prior to bone maturation.

Moreover, in some embodiments, methods of promoting and/or accelerating bone regeneration described herein can comprise maintaining the graft or scaffold in the bone site for a period of time after disposing the graft or scaffold in the bone growth site. Any period of time not inconsistent with the objective of the present disclosure can be used. For example, in some cases, the graft or scaffold can be maintained for at least 1 month, such as for at least 3 months, at least 6 months, at least 9 months, or at least 12 months. In certain embodiments, a graft or scaffold may degrade or biodegrade within the bone site. In such embodiments, maintenance of the graft or scaffold can comprise or include maintaining the graft or scaffold until a desired portion of the graft or scaffold has degraded or biodegraded. For example, methods can comprise maintaining the graft or scaffold in the bone site until at least 50%, at least 60%, at least 70%, at least 80%, at least 90%, at least 95%, or at least 99% of the graft or scaffold has degraded or biodegraded. In certain embodiments, methods can comprise maintaining the graft or scaffold in the bone site until all or substantially all of the graft or scaffold has degraded or biodegraded. In some embodiments, biodegradation of the graft or scaffold can be measured by measuring the fluorescence intensity at the time of delivery and comparing additional fluorescence intensity measurements at later times to the time of delivery measurement.

In another aspect, this disclosure describes a method for making xylitol doped poly(octamethylene citrate) (POC) polyesters and films, porous scaffolds and composites of the same. Xylitol is incorporated into the polymer via esterification. Xylitol doped polymers can be formed into films through solvent casting followed by further crosslinking via thermal esterification, porous scaffolds via physical mixing of polymer solutions with sodium chloride or other porogen and subsequent thermal crosslinking and porogen leaching, and composites via physical mixing of polymer with hydroxyapatite or other filers, molding and subsequent thermal crosslinking.

In another aspect, the compositions and methods of this disclosure incorporate xylitol homogenously into POC though chemical reaction.

In another aspect, the compositions of this disclosure increase the mechanical strength and degradation rate of POC films in dry and hydrated conditions through xylitol doping. Additionally, this compositions and methods of this disclosure tune the degradation rate of materials independently of mechanical properties through xylitol doping.

In another aspect, the compositions and methods of this disclosure fabricate porous scaffolds and composites with homogenous physical properties and improved mechanical strength utilizing xylitol doped POC.

In another aspect, the compositions and methods of this disclosure fabricate materials capable of promoting osteogenic differentiation of human mesenchymal stem cells using xylitol doped POC.

In another aspect, the compositions and methods of the disclosure fabricate materials with antibacterial capability using xylitol doped POC.

In another aspect, the compositions and methods of the disclosure fabricate materials with antioxidant and immunomodulatory capability through xylitol doping of citrate-based materials.

In another aspect, the compositions and methods of the disclosure incorporate xylitol doping into various citrate based materials including but not limited to poly(octamethylene citrate) (POC), biodegradable photoluminescent polymer (BPLPs), and injectable citrate based mussel inspired bioadhesives (iCMBAs).

In another aspect, the compositions and methods of the disclosure fabricate stimuli responsive self-healing citrate-based materials utilizing xylitol doping.

In another aspect, the compositions and methods of the disclosure create photoluminescent materials through xylitol doping of citrate-based materials.

In another aspect, the compositions and methods of the disclosure create materials with controlled and tunable release of bioactive factors (citrate and xylitol) for synergistic biological activity through xylitol doping of citrate-based materials.

Further applications of the compositions described herein include but are not limited to the following: orthopedic tissue engineering materials including composites and porous scaffolds for critical size segmental defect repair and fixation and spinal fusion and films for periosteum repair and barrier functionality; porous scaffolds for wound dressing applications; antibacterial materials; antioxidant materials; antiresorptive materials for osteoporosis treatment; self-healing materials; and injectable materials for void filling and fracture fixation.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for.

Results described further herein demonstrate that varying the ratio of xylitol within POC/HA compositions provides for homogenous increases in mechanical properties (exceeding that of native bone tissue) while modulating the biodegradation rate significantly. Thus, incorporating of xylitol into citrate-based materials results in improved compositions useful as tissue engineering materials through enhanced physical and biological properties. For example, methods disclosed herein provide for homogenous incorporation of xylitol into POC via xylitol doping. The homogenous incorporation of xylitol into POC provides for compositions with increased mechanical strength and improved (quicker and more controllable) biodegradation rate, as compared to traditional POC compositions. The increased mechanical strength and improved biodegradation is exhibited in both dry and hydrated conditions. Additionally, the biodegradation rate of composite materials is tunable. It is important to note that the tunability of the biodegradation rate is independent of mechanical properties, i.e., the biodegradation rate can be tuned with little to no change in mechanical properties.

Examples of methods disclosed herein involve fabricating xylitol doped POC materials (e.g., polymers, films, scaffolds, and compositions, etc.). Polymers other than POC can be used, such a biodegradable photoluminescent polymers (BPLPs), injectable citrate-based mussel inspired bioadhesives (iCMBA), etc. Xylitol can be incorporated into the polymer via esterification. In one representative example, citric acid and octanediol/xlitol with a 1:1 mole ratio can be melted at 160° C. under stirring for ten minutes. The reaction temperature can then be reduced to 140° C., wherein the reaction proceeds until the pre-polymer can no longer be stirred due to viscosity, at which point the reaction may be quenched with dioxane. Following polymerization, the pre-polymer can be purified by precipitation in deionized water, lyophilized, and dissolved in organic solvent to form pre-polymer solutions.

Xylitol doped citrate-based polyesters may be synthesized via the above general procedure using a variety of diols. Suitable diols can be small molecule diols such as 1,2-ethylene glycol, 1,3-propanediol, 1,4-butanediol, 1,5-pentanediol, 1,6-hexanediol, 1,8-octanediol, 1,10-decanediol, and 1,12-dodecanediol or macrodiols such as poly(ethylene glycol) (PEG) or combinations thereof. Xylitol doped polymers may be synthesized with citrate:diol+xylitol ratios of 1.5:1 to 1:1.5. Xylitol doped polymers may be synthesized with varying xylitol contents from greater than 0 to less than 100% diol substitution.

Xylitol doped polymers can be formed into films through solvent casting followed by further crosslinking via thermal esterification. For instance, xylitol doped POC films can be prepared by casting prepolymer solutions in Teflon dishes, followed by solvent evaporation and thermal crosslinking.

Xylitol doped polymers can be formed into porous scaffolds via physical mixing of polymer solutions with sodium chloride or other porogen and subsequent thermal crosslinking and porogen leaching. For instance, xylitol doped POC porous scaffolds can be prepared by mixing pre-polymer solutions with porogen until a paste is formed, which can then be packed into Teflon dishes and thermally crosslinked. Salt can be leached by immersion in DI water followed by lyophilization.

Xylitol doped polymers can be formed into composites via physical mixing of polymer with hydroxyapatite or other fillers, molding, and subsequent thermal crosslinking. For instance, xylitol doped POC compositions can be formed by mixing pre-polymers with filler materials until a clay-life consistency is achieved, followed my molding into the desired shape and thermal crosslinking. Examples of filler materials include but are not limited to hydroxyapatite, B-tricalcium phosphate, pearl powder, octacalcium phosphate, etc.

Referring now to Tables 1 and 2, xylitol doped compositions were prepared both with a stoichiometric balance of —COOH and —OH functional groups among the monomers and with imbalanced ratios (favoring excess —OH groups with increased xylitol content). Excess —OH groups resulted in increased hydrogen bond interactions. In the case of the synthesized polymers, excess xylitol based —OH clusters led to areas of hydrogen bonding while still allowing crosslinking to proceed. Stoichiometrically balanced formulations led to polymers requiring extremely lengthy crosslinking times to achieve appreciable results. In a few cases where crosslinking was successful (NX1 and NX3), mechanics compared unfavorably with the corresponding unbalance formulation.

TABLE 1
Mole Ratio of Citric Acid: (Octanediol + Xylitol)
CXBE Formulations

| | Citric Acid (mols) | Xylitol (mols) | Octanediol (mols) |
|---|---|---|---|
| POC | 0.11 | 0 | 0.11 |
| X1 | 0.11 | 0.01 | 0.10 |
| X3 | 0.11 | 0.03 | 0.08 |
| X5 | 0.11 | 0.05 | 0.06 |
| X6 | 0.11 | 0.06 | 0.05 |
| X8 | 0.11 | 0.08 | 0.03 |

TABLE 2
1:1 Mole Ratio of —COOH:—OH
CXBE Formulations

| | Citric Acid (mols) | Xylitol (mols) | Octanediol (mols) |
|---|---|---|---|
| NX1 | 0.125 | 0.01 | 0.10 |
| NX3 | 0.155 | 0.03 | 0.08 |
| NX5 | 0.185 | 0.05 | 0.06 |
| NX6 | 0.20 | 0.06 | 0.05 |
| NX8 | 0.23 | 0.08 | 0.03 |

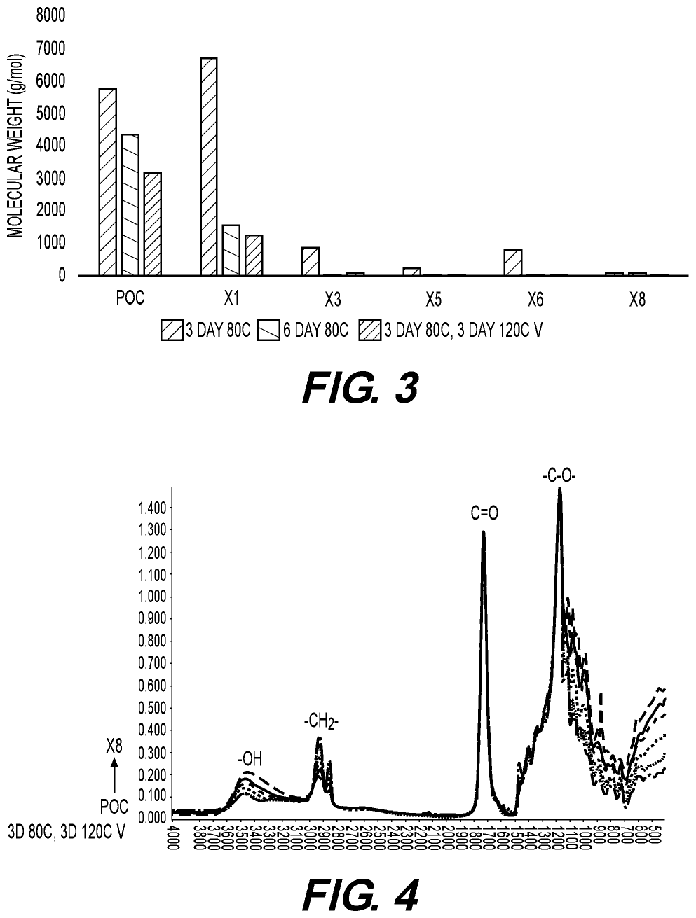

Referring to Table 3 and FIGS. 2 and 3, high strength, rapidly degradable polymer can be engineered by simultaneously increasing crosslinking density and hydrophilicity via xylitol incorporation. Incorporation of increasing amounts of xylitol leads to: decreased molecular weight, increasing polymer density, and vastly decreased molecular weight between crosslinks. Overall, results indicate formation of a highly branched and highly crosslinked polymer network, leading to increased mechanics while maintaining degradability due to the hydrophilic nature of xylitol.

TABLE 3
Molecular Weights of POC-Xylitol

| Xylitol (mols) | Mₙ | M_{w} | PDI |
|---|---|---|---|
| POC (control) | 1474 | 1624 | 1.10 |
| 0.01 | 1404 | 1543 | 1.10 |
| 0.03 | 1280 | 1377 | 1.08 |
| 0.05 | 1181 | 1257 | 1.06 |
| 0.06 | 1142 | 1206 | 1.06 |
| 0.08 | 1141 | 1198 | 1.05 |

Referring to FIG. 4, Fourier-transform infrared spectra of the compositions described above were obtained. An increased —OH signal was observed with increased levels of xylitol content within the polymer, indicating the formation of hydrogen bonds between polymer chains. This is further demonstrated by the broad slope of the —OH signal from 3300-3400. Such hydrogen bonds reinforce polymer mechanics.

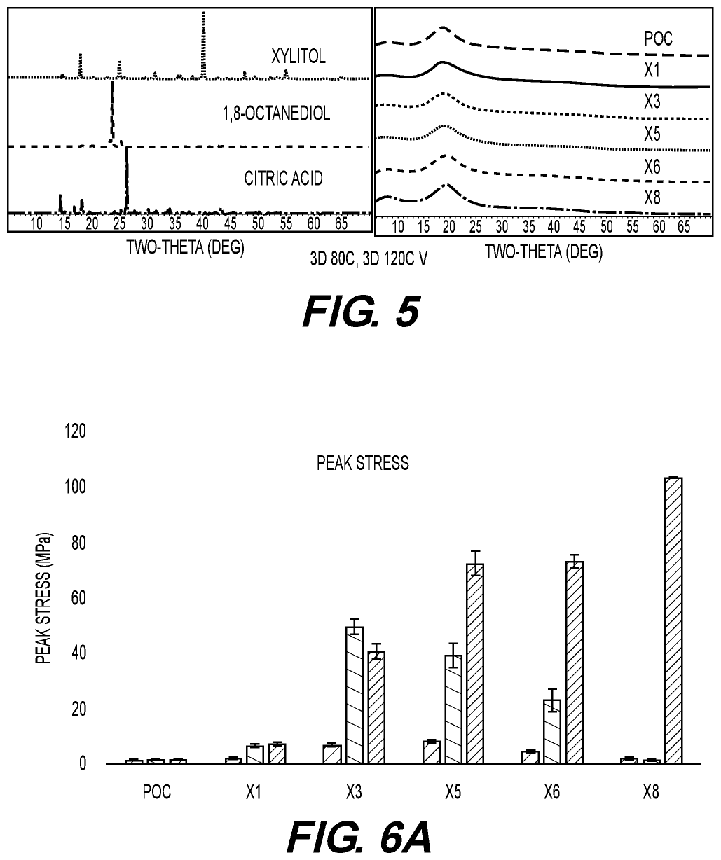

Referring to FIG. 5, x-ray diffraction spectra for the compositions described above were obtained. The spectra depict a lack of crystallinity of the polymers with increasing xylitol content.

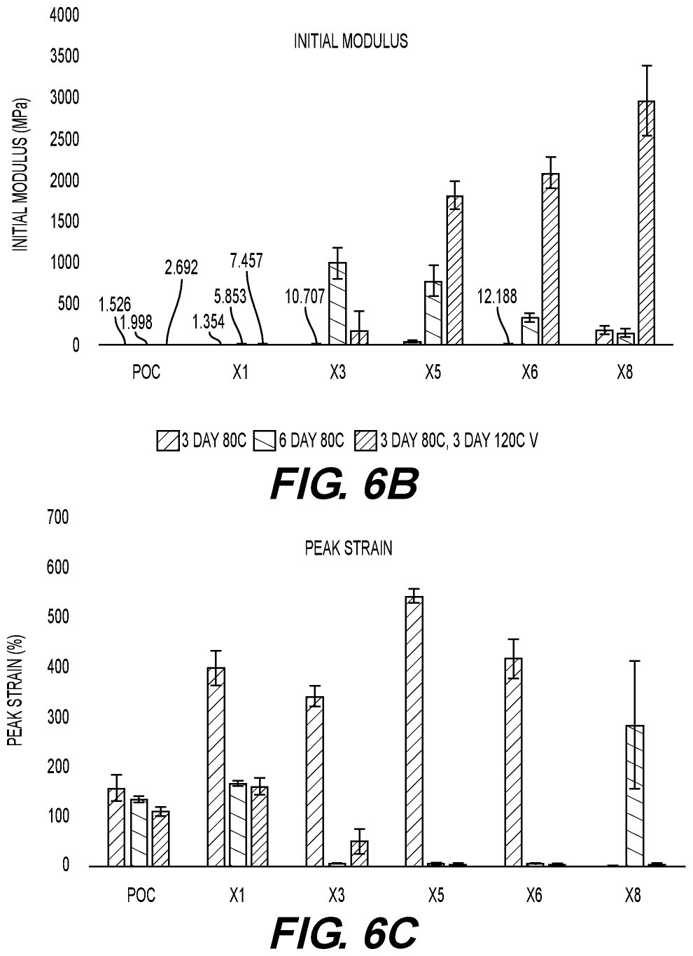

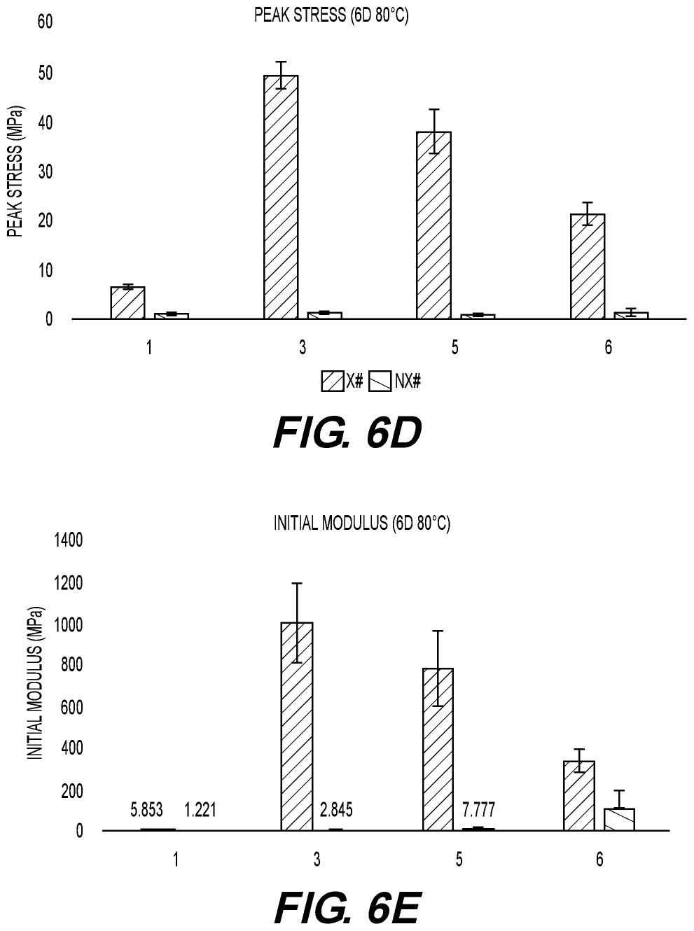

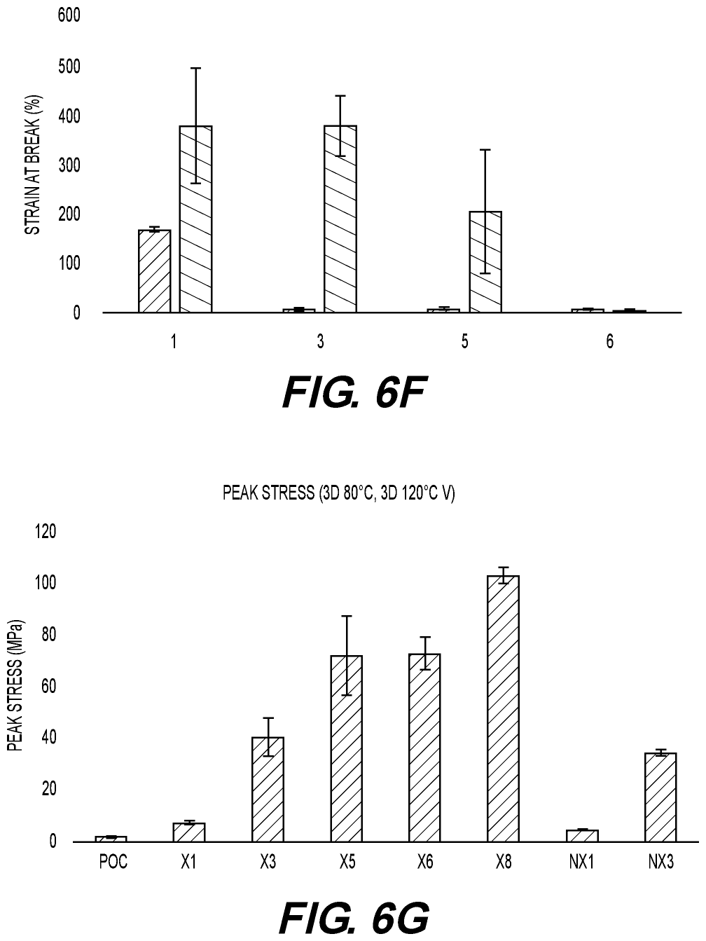

Referring to FIGS. 6A-6G, polymer films were prepared from the compositions described above to analyze tensile film mechanics. Notably, formulations above NX3 could not be crosslinked under the conditions used. The obtained measurements demonstrate the tunability of film mechanics in a manner that is capable of matching a range of biological tissues (such as skin, nerve, bone, etc.).

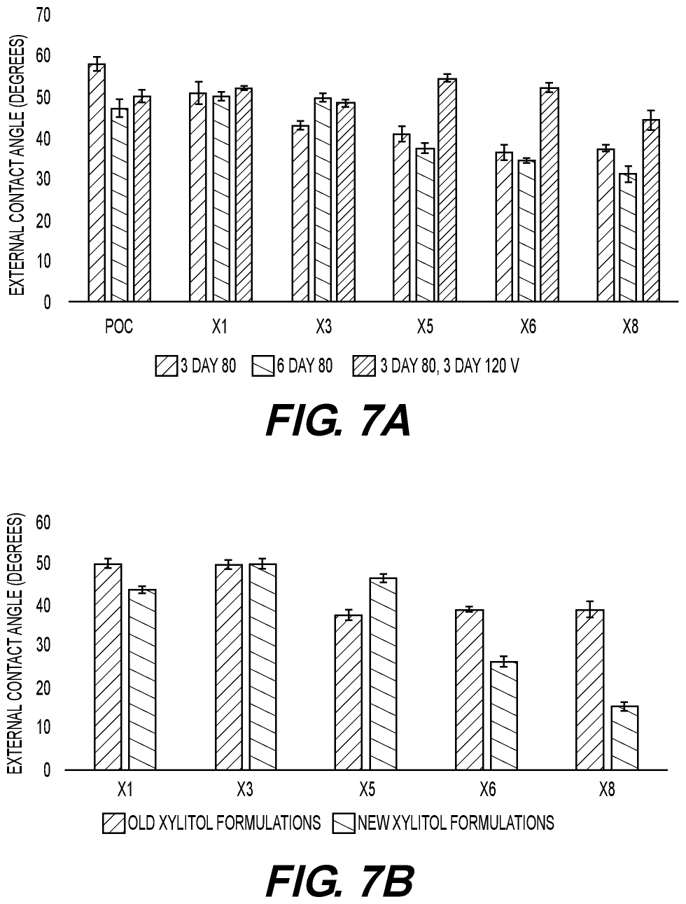

Referring to FIGS. 7A and 7B, the external contact angle for the compositions described above was measured. The observed contact angles demonstrate the hydrophilicity of the representative materials.

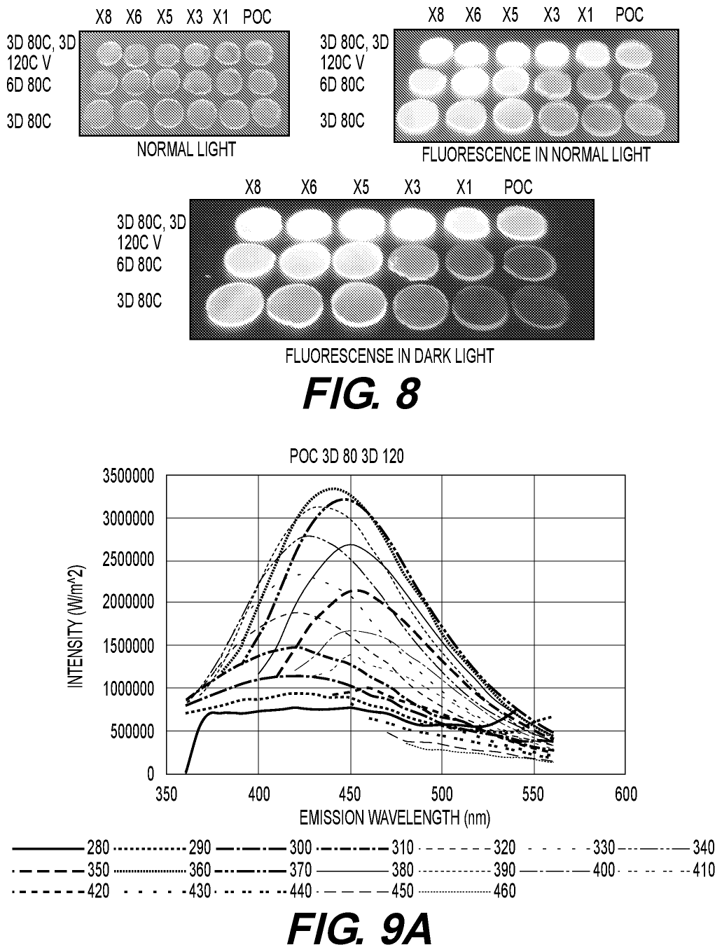

Referring to FIG. 8, the fluorescence of the prepared films was analyzed. Enhanced fluorescence was observed with increasing xylitol content. Increased branching and crosslink density with increasing xylitol content leads to increased hydrogen bond interactions between —OH and —C═O groups (pi-pi* and n-sigma* interactions), and thus increased fluorescence.

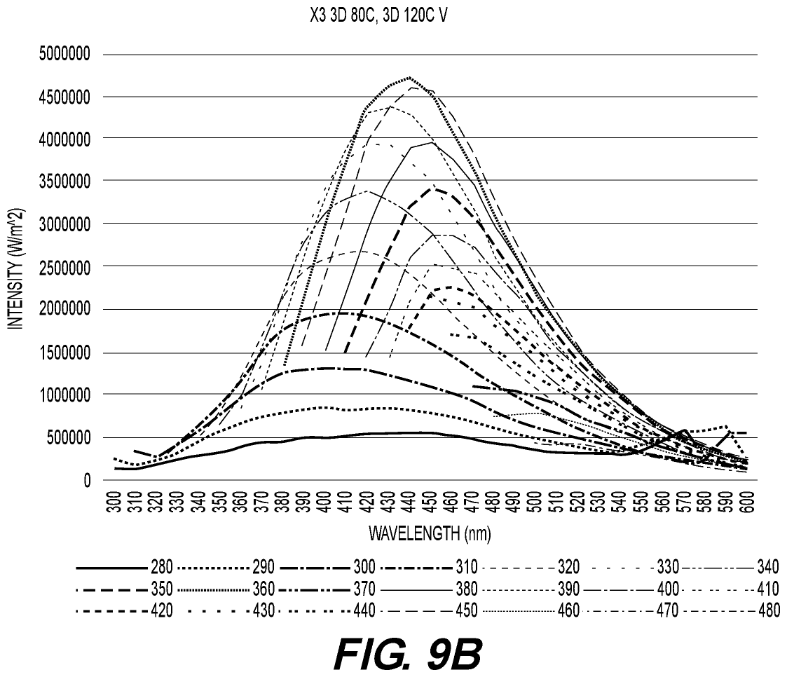

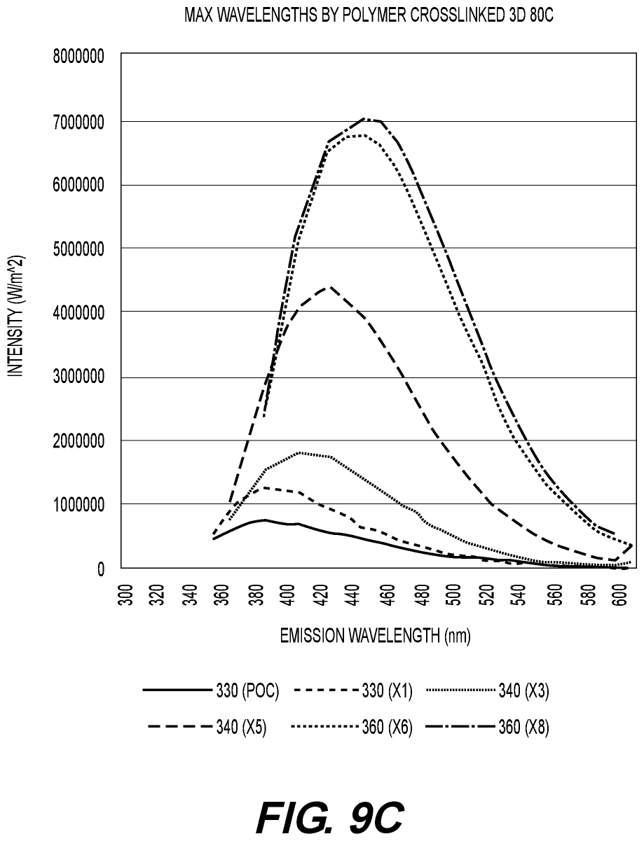

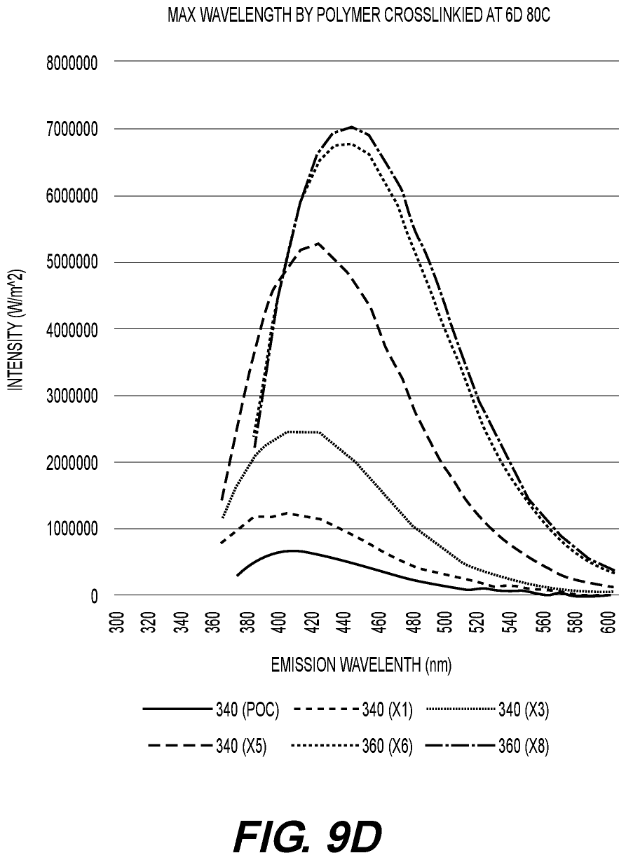

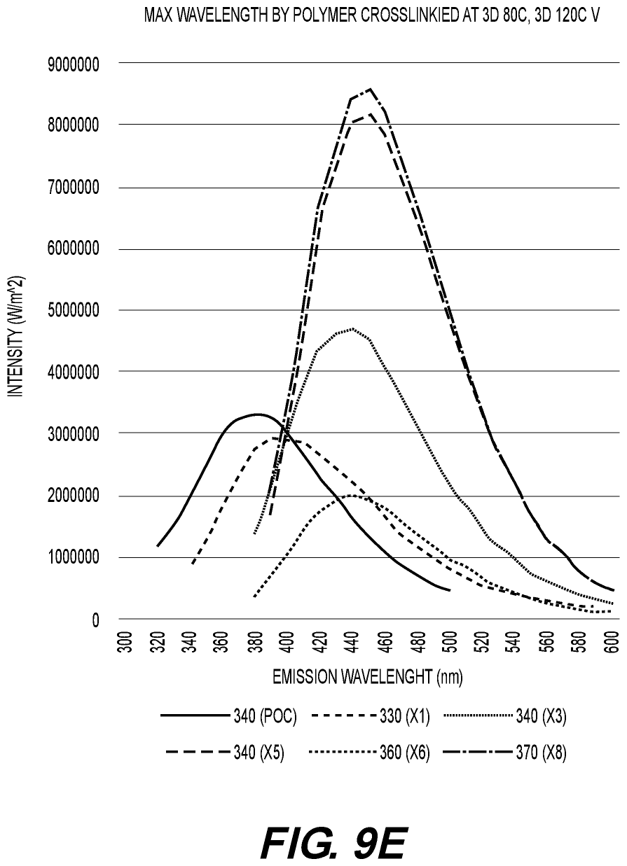

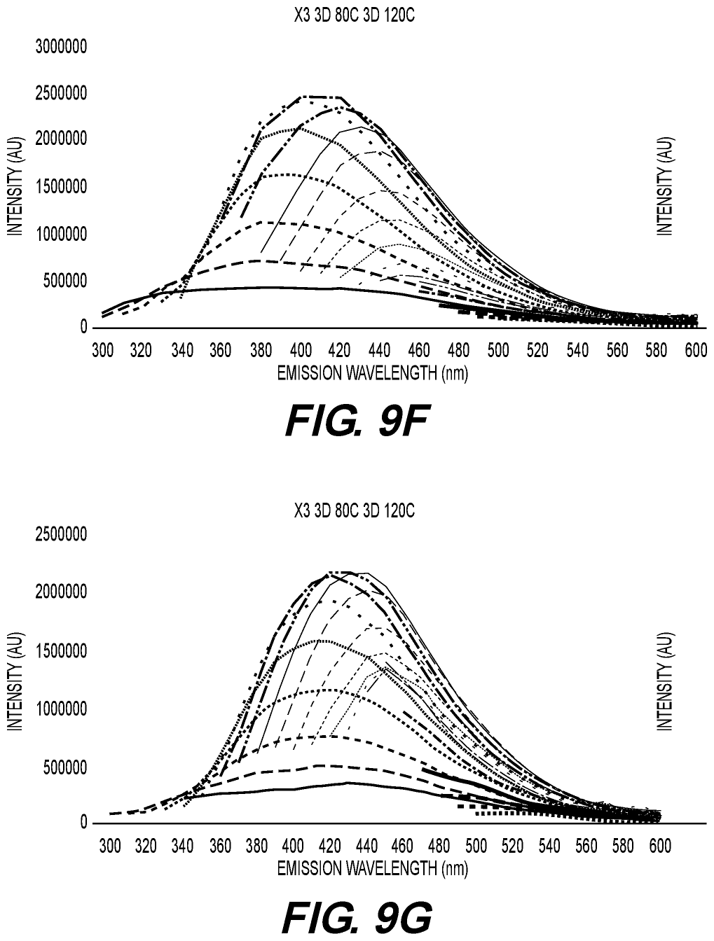

Referring to FIGS. 9A-9G, fluorescence emission spectra were obtained for the above-prepared compositions. These spectra show that the disclosed compositions may be useful for imaging and light delivery in vivo.

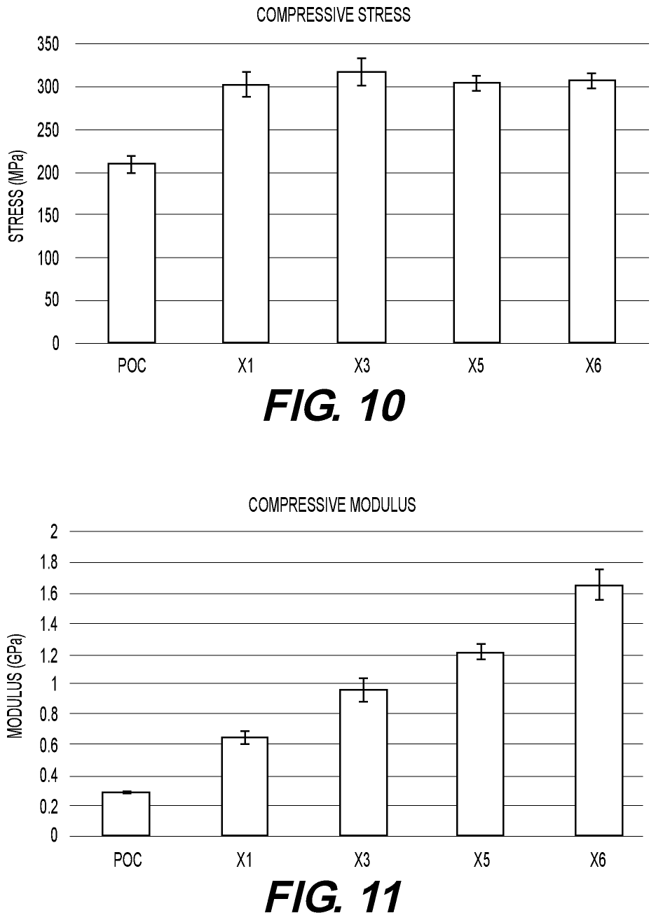

Referring now to FIG. 10, composites were prepared of the compositions described above and 60 weight percent hydroxyapatite (HA), and compressive mechanical properties of these compositions were analyzed. The obtained data demonstrate that uniform stress on the composites regardless of the xylitol content. Further, xylitol incorporation did not diminish the ability to incorporate HA, presumably due to the ability of xylitol to chelate ions.

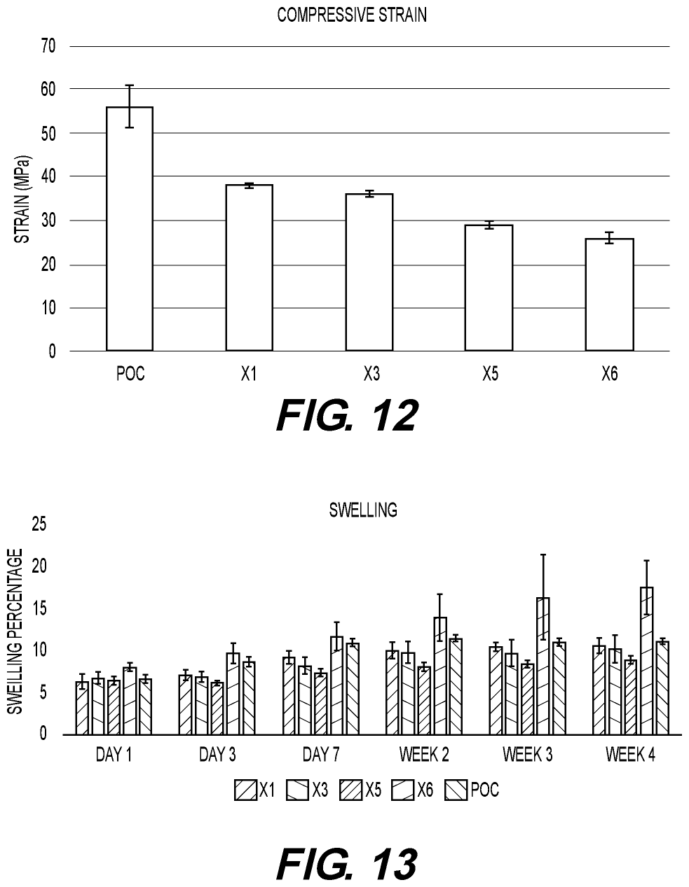

Referring now to FIG. 11, the compressive modulus of the prepared composites was analyzed. The values obtained were significantly enhanced compared to composites lacking xylitol. Measurements of compressive strain were also obtained (see FIG. 12).

Referring now to FIG. 13, the percentage of swelling for the prepared composites was analyzed. Composites containing xylitol were found to swell at the same rate as composites lacking xylitol despite the increased hydrophilic character of xylitol as a monomeric component.

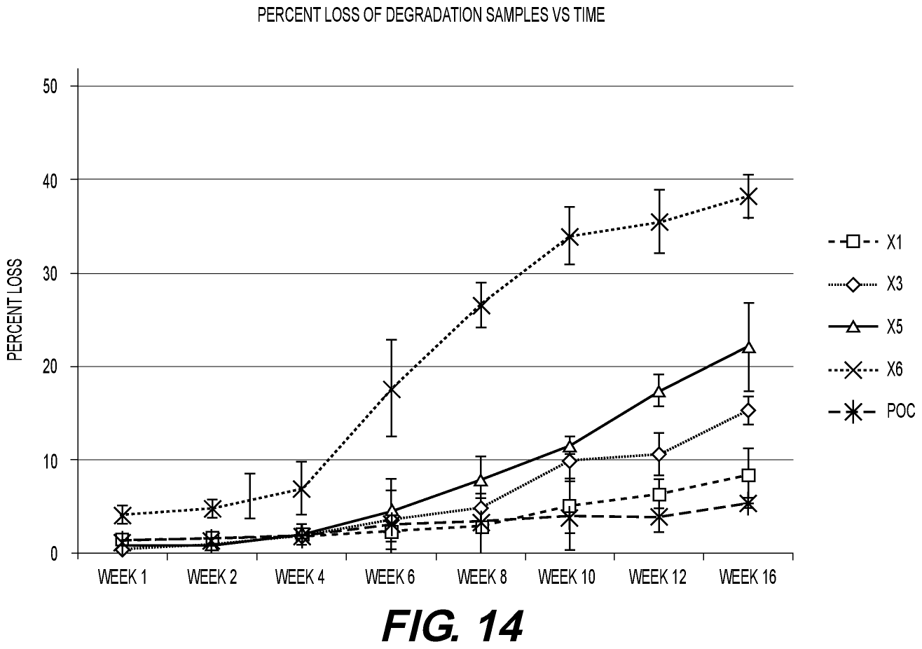

Referring now to FIG. 14, the degradation (in percent loss) of the disclosed compositions was analyzed over time. The compositions were found to have a tunable degradation rate of 5% to 40% over 16 weeks. Incorporating of higher amounts of xylitol led to complete loss of polymer weight (˜40%) in four months. Critically, the degradation rate can be tuned without negatively impacting or even significantly changing the initial mechanics of the composition.

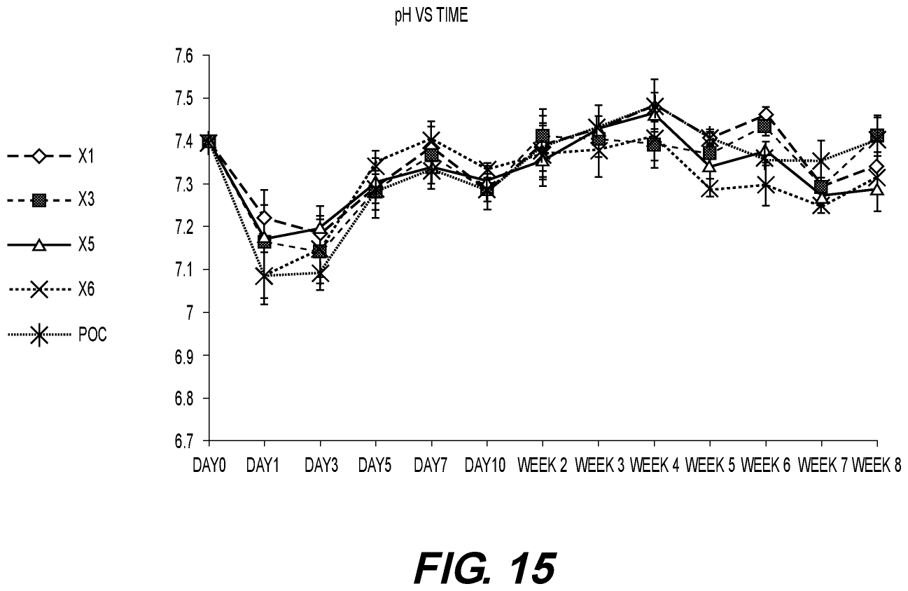

Referring now to FIG. 15, the pH of the composites over time was analyzed. A return to physiological pH (˜7.4) was observed within one week. Critically; an acute drop in pH is associated with normal bone healing while a prolonged acidic environment is indicative of disease states or abnormal bone healing; xylitol containing composites are capable of replicating a desired pH profile for the bone environment.

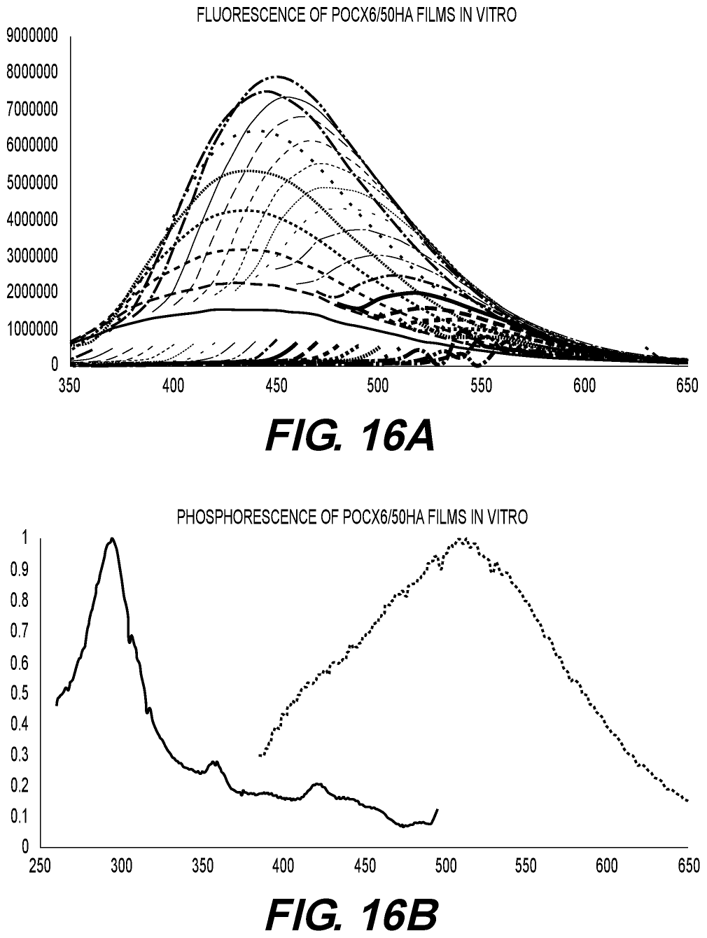

Referring to FIGS. 16A and 16B, fluorescence and room temperature phosphorescent spectra were obtained for the above-described composites. The presence of room temperature phosphorescence demonstrates that these composites may be used in multiple imaging modalities. In particular, phosphorescence may be used preferentially in vivo to avoid the autofluorescence of biological tissues through the intrinsic delayed emission of phosphorescence versus fluorescence.

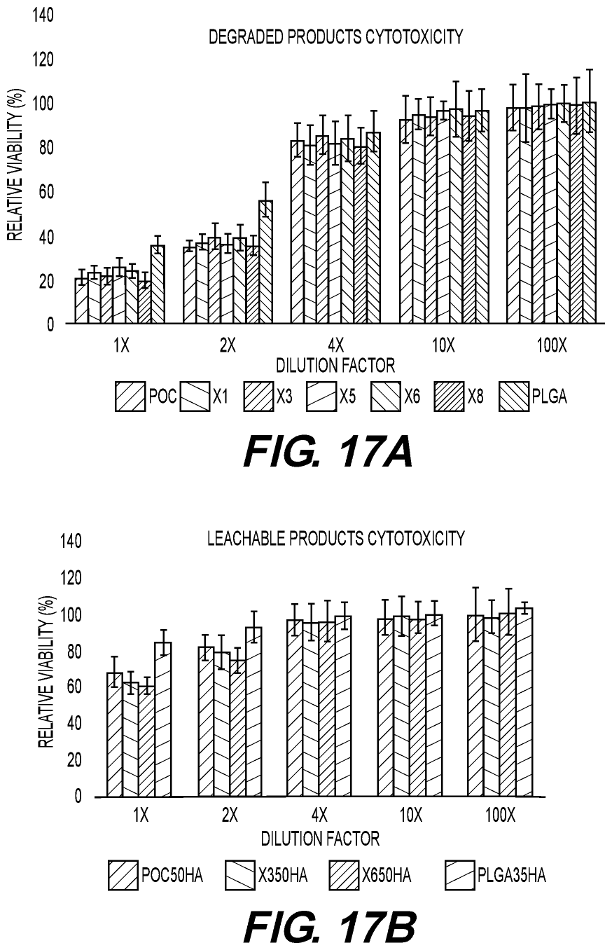

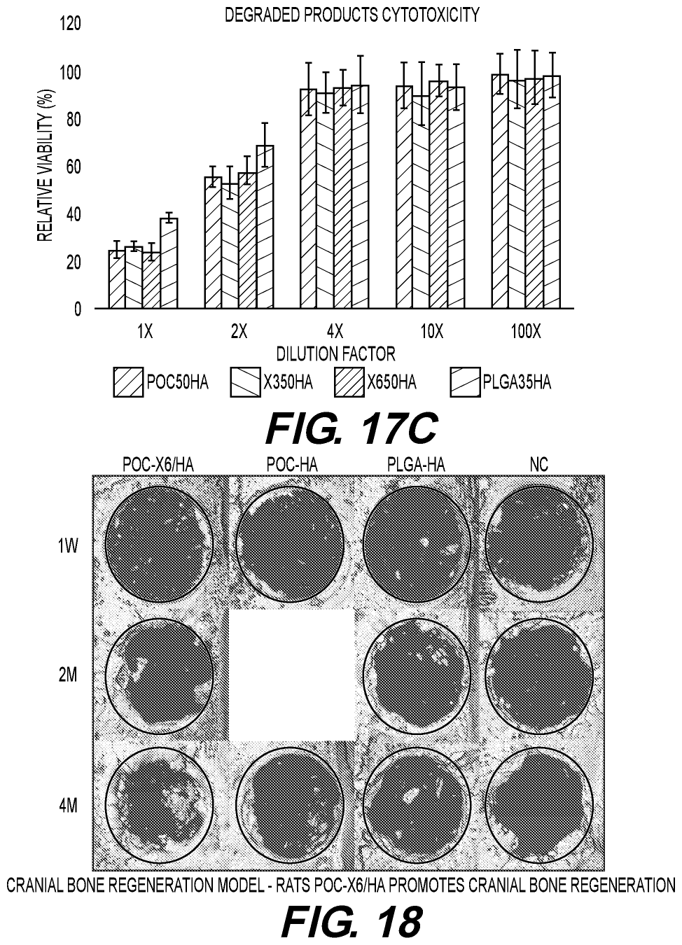

Referring to FIGS. 17A-17C, the in vitro cytotoxicity of the film degradation products and both the composite leachables and degradation products were evaluated against MG63 cells.

Referring to FIG. 18, the disclosed composites (POC-X6/HA) demonstrated cranial bone regeneration that was similar to that found for ALGA/HA materials used in the clinic.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A composition comprising a polymer or oligomer formed from one or more monomers of Formula (A1), one or more monomers independently selected from Formula (B1) and Formula (B2), and one or more monomers of Formula (C1): 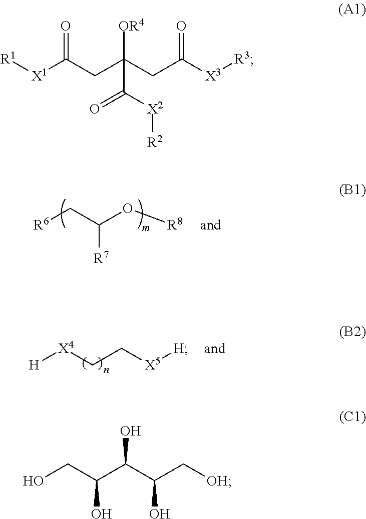
wherein:
X¹, X², and X³ are each independently —O— or —NH—;
X⁴ and X⁵ are independently —O— or —NH;
R¹, R², and R³ are each independently —H, C₁-C₂₂ alkyl, C₂-C₂₂ alkenyl, or M⁺;
R⁴ is H or M⁺;
R⁶ is —H, —NH, —OH, —OCH₃, —OCH₂CH₃; —CH₃, or —CH₂CH₃;
R⁷ is —H, C₁-C₂₃ alkyl, or C₂-C₂₃ alkenyl;
R⁸ is —H, C₁-C₂₃ alkyl, C₂-C₂₃ alkenyl, —CH₂CH₂OH, or —CH₂CH₂NH₂;
n and m are independently integers ranging from 1 to 2000; and
M⁺ is a cation.

2. The composition of claim 1, wherein X¹, X², and X³ are each —O—.

3. The composition of claim 1, wherein R⁴ is —H.

4. The composition of claim 1, wherein the one or more monomers of Formula (A1) comprise citric acid or a citrate.

5. The composition of claim 1, wherein the one or more monomers of Formula (B1) are selected from poly(ethylene glycol) and poly(propylene glycol).

6. The composition of claim 1, wherein the one or more monomers of Formula (B2) are selected from 1,2-ethylene glycol, 1,3-propanediol, 1,4-butanediol, 1,5-pentanediol, 1,6-hexanediol, 1,8-octanediol, 1,10-decanediol, and 1,12-dodecanediol.

7. The composition of claim 1, wherein the one or more monomers independently selected from Formula (B1) and Formula (B2) and the one or more monomers of Formula (C1) are present in a molar ratio ranging from about 20:1 to about 1:20.

8. The composition of claim 1, wherein the polymer or oligomer is further formed from one or more monomers of Formula (D1), Formula (E1), Formula (E2), Formula (E3), Formula (E4), Formula (F1), Formula (F2), Formula (G1), Formula (H1), Formula (H2), Formula (H3), Formula (I1), Formula (I2), Formula (I3), Formula (I4), Formula (I5), and Formula (I6): 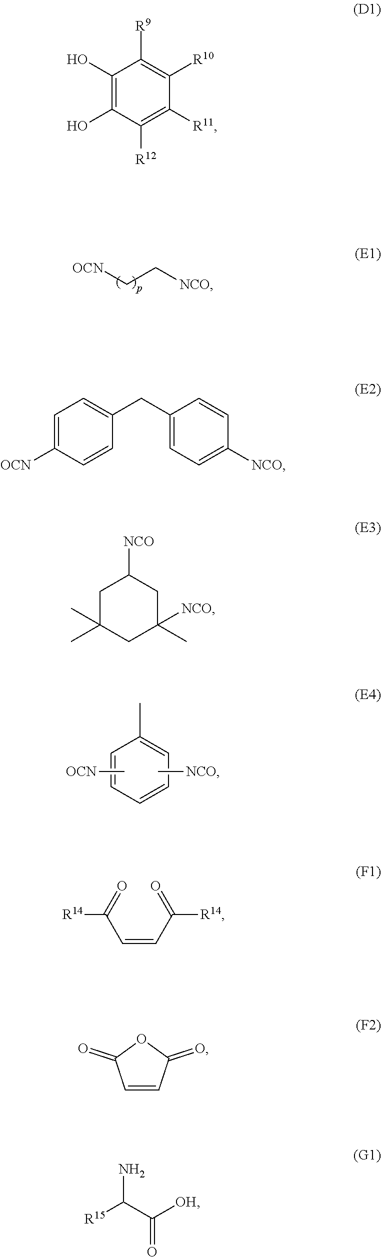 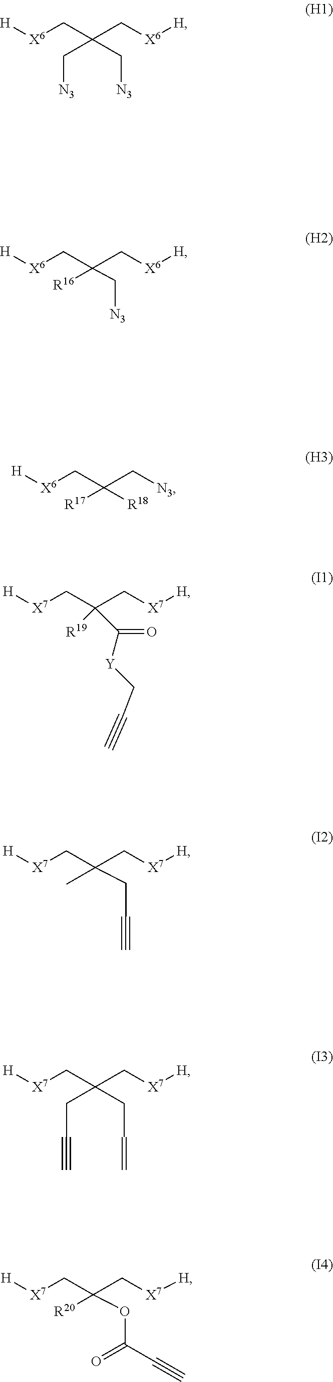 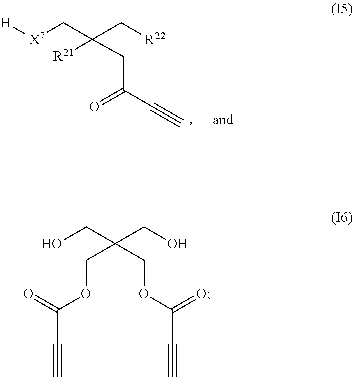
wherein:
R⁹, R¹⁰, R¹¹, and R¹² are each independently selected from —H, —OH, —CH₂(CH₂)ₓNH₂, —CH₂(CHR¹³)NH₂, —CH₂(CH₂)ₓOH, —CH₂(CHR¹³)OH, and —CH₂(CH₂)ₓCOOH;
R¹³ is —COOH or —(CH₂)_{y}COOH;
x and y are independently an integer ranging from 1 to 10;
p is an integer ranging from 1 to 20;
R¹⁴ is selected from —OH, —OCH₃, —OCH₂CH₃, and —Cl;
R¹⁵ is an amino acid side chain;
X⁶ is independently selected at each occurrence from —O— or —NH—;
R¹⁶ is —CH₃ or —CH₂CH₃;
R¹⁷ and R¹⁸ are each independently —CH₂N₃, —CH₃, or —CH₂CH₃;
X⁷ and Y are independently —O— or —NH—;
R¹⁹ and R²⁰ are each independently —CH₃ or —CH₂CH₃;
R²¹ is —OC(O)CCH, —CH₃, or —CH₂CH₃; and
R²² is —CH₃, —OH, or —NH₂.

9-14. (canceled)

15. The composition of claim 1, wherein the polymer or oligomer is thermally crosslinked.

16. (canceled)

17. The composition of claim 1, having a tensile strength of about 1 MPa to about 120 MPa in a dry state, and/or having a tensile modulus of about 1 MPa to about 3.5 GPa in a dry state.

18. (canceled)

19. The composition of claim 1, wherein the composition is luminescent.

20. The composition of claim 1, further comprising an inorganic material.

21. (canceled)

22. (canceled)

23. The composition of claim 20, wherein the composition has a compressive strength ranging from about 250 MPa to about 350 MPa, and/or wherein the composition has a compressive modulus ranging from about 100 KPa to about 1.8 GPa.

24. (canceled)

25. (canceled)

26. The composition of claim 1, further comprising an antioxidant, pharmaceutically active agent, biomolecule, or cell.

27. The composition of claim 1, configured to degrade in less than 4 months.

28. A method of promoting and/or accelerating bone regeneration comprising:
delivering a composition of claim 1 to a bone site.

29. The method of claim 28, wherein the composition is delivered before and/or during a proliferation stage of osteogenesis at the bone site.

30. The method of claim 28, further comprising delivering stem cells to the bone site.

31. The method of claim 28, wherein the bone site is an intramembranous ossification site or an endochondral ossification site.

32. (canceled)

33. A method of preparing a composition of claim 1 comprising:
polymerizing a polymerizable composition to form the composition, the polymerizable composition comprising one or more monomers of Formula (A1), one or more monomers independently selected from Formula (B1) and Formula (B2), and one or more monomers of Formula (C1): 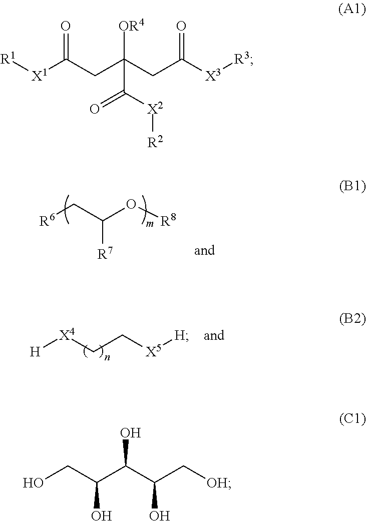
wherein:
X¹, X², and X³ are each independently —O— or —NH—;
X⁴ and X⁵ are independently —O— or —NH;
R¹, R², and R³ are each independently —H, C₁-C₂₂ alkyl, C₂-C₂₂ alkenyl, or M⁺;
R⁴ is H or M⁺;
R⁶ is —H, —NH, —OH, —OCH₃, —OCH₂CH₃; —CH₃, or —CH₂CH₃;
R⁷ is —H, C₁-C₂₃ alkyl, or C₂-C₂₃ alkenyl;
R⁸ is —H, C₁-C₂₃ alkyl, C₂-C₂₃ alkenyl, —CH₂CH₂OH, or —CH₂CH₂NH₂;
n and m are independently integers ranging from 1 to 2000; and
M⁺ is a cation.

34-52. (canceled)
